SEQUENCE LISTING (68 sequences)

SEQ ID NO: 1 - protein, 1260 aa, Clostridium bifermentans
MLQIRVFNYNDPIDGENIVELRYHNRSPVKAFQIVDGIWIIPERYNFTNDTKKVPDDRALTILEDEVFAVRENDYLTTDVNEKNSFLNNITKLFKRINSSNIGNQLLNYISTSVPYPVVSTNSIKARDYNTIKFDSIDGRRITKSANVLIYGPSMKNLLDKQTRAINGEEAKNGIGCLSDIIFSPNYLSVQTVSSSRFVEDPASSLTHELIHALHNLYGIQYPGEEKFKFGGFIDKLLGTRECIDYEEVLTYGGKDSEIIRKKIDKSLYPDDFVNKYGEMYKRIKGSNPYYPDEKKLKQSFLNRMNPFDQNGTFDTKEFKNHLMDLWFGLNESEFAKEKKILVRKHYITKQINPKYTELTNDVYTEDKGFVNGQSIDNQNFKIIDDLISKKVKLCSITSKNRVNICIDVNKEDLYFISDKEGFENIDFSEPEIRYDSNVTTATTSSFTDHFLVNRTFNDSDRFPPVELEYAIEPAEIVDNTIMPDIDQKSEISLDNLTTFHYLNAQKMDLGFDSSKEQLKMVTSIEESLLDSKKVYTPFTRTAHSVNERISGIAESYLFYQWLKTVINDFTDELNQKSNTDKVADISWIIPYVGPALNIGLDLSHGDFTKAFEDLGVSILFAIAPEFATISLVALSIYENIEEDSQKEKVINKVENTLARRIEKWHQVYAFMVAQWWGMVHTQIDTRIHQMYESLSHQIIAIKANMEYQLSHYKGPDNDKLLLKDYIYEAEIALNTSANRAMKNIERFMIESSISYLKNNLIPSVVENLKKFDADTKKNLDQFIDKNSSVLGSDLHILKSQVDLELNPTTKVAFNIQSIPDFDINALIDRLGIQLKDNLVFSLGVESDKIKDLSGNNTNLEVKTGVQIVDGRDSKTIRLNSNENSSIIVQKNESINFSYFSDFTISFWIRVPRLNKNDFIDLGIEYDLVNNMDNQGWKISLKDGNLVWRMKDRFGKIIDIITSLTFSNSFIDKYISSNIWRHITITVNQLKDCTLYINGDKIDSKSINELRGIDNNSPIIFKLEGNRNKNQFIRLDQFNIYQRALNESEVEMLFNSYFNSNILRDFWGEPLEYNKSYYMINQAILGGPLRSTYKSWYGEYYPYISRMRTFNVSSFILIPYLYHKGSDVEKVKIINKNNVDKYVRKNDVADVKFENYGNLILTLPMYSKIKERYMVLNEGRNGDLKLIQLQSNDKYYCQIRIFEMYRNGLLSIADDENWLYSSGWYLYSSGWYLDNYKTLDLKKHTKTNWYFVSEDEGWKE

SEQ ID NO: 2 - DNA, 3783 bp, Clostridium bifermentans
atgctacaaataagagtttttaattataatgatccaattgatggagaaaatatcgtggagttaagataccataacaggagccctgtaaaagcatttcaaatagtagatggtatatggataattccagaaagatataactttacaaacgatacaaaaaaagttccagacgatcgagctcttactattctggaagatgaagtttttgctgttcgcgaaaatgactatttaacaacagatgttaatgaaaaaaattcctttttaaataatattactaagctttttaagcgtattaattcaagtaacattggtaatcagttacttaattatatttcaacaagcgtcccatatccagttgtgagtacaaattcaataaaggctagagactataatacaattaaatttgattcaattgatgggcgaagaattacaaaatctgcaaatgtacttatctacggaccaagtatgaaaaatttactagataaacaaacaagggctatcaatggggaagaagcaaaaaatggtataggatgtttaagtgatattattttttctccaaattacttatctgtccaaactgtttcttcaagtaggtttgttgaagatcctgcatcatcacttacacatgaacttatccatgccttacataatttatatggaatacaatatcctggagaagaaaaatttaaatttggaggatttattgataaactattaggaactagagaatgcatagattatgaggaagtcttaacatatggaggaaaagattccgaaattataagaaagaaaattgataagtccttatatcctgatgattttgtaaataagtatggtgaaatgtataagcgtataaaaggatctaatccttattatcccgacgaaaaaaaattaaaacaaagttttttaaacagaatgaatccatttgatcaaaatggaacttttgatactaaagaatttaaaaatcatcttatggatttatggtttgggttaaatgagagtgaatttgctaaagaaaagaagattttagtcagaaagcactatataacaaagcaaattaatcctaaatacacagaacttactaatgatgtatatactgaagataaaggctttgtaaatggtcaatctatagacaatcaaaattttaaaataattgatgatttaatatcaaaaaaagtaaaactatgttctataacatctaaaaatcgagtaaatatttgtatagacgttaataaagaagatttatatttcataagtgataaagaaggttttgaaaatatagatttttccgagccggaaattagatatgatagtaatgtaactacagcaactacctcttcttttacagaccattttttagtaaatagaacttttaacgatagtgatagatttccacctgtagaattagaatatgctatcgaaccagctgaaatagttgataacactataatgccagatattgatcaaaaaagcgaaatatctctcgataacttaacgacctttcactatttaaatgctcaaaaaatggatttgggatttgattcatcaaaagaacagttaaagatggttacatcaatagaggaatcattattagattcaaaaaaggtatacacaccatttacgagaactgcacatagtgtaaatgaacgtatatctggaatagcggaaagttacttattttatcaatggttaaaaactgttataaatgattttacagatgaattaaaccaaaagagtaatactgacaaagttgctgatatttcttggattataccctatgttggacctgctttaaatattggccttgatttatctcatggagattttactaaagcttttgaagatttaggggtttctattttatttgctattgctccagaatttgcaactataagtcttgtagctctttcaatatatgaaaatatagaagaggattcacaaaaagaaaaagtaattaataaagtagaaaatacattagcaaggagaatagaaaaatggcaccaagtttatgctttcatggtggctcagtggtggggtatggttcatactcagatagacactagaattcatcaaatgtatgaatcactttctcatcaaattatagcaattaaagctaatatggagtatcagttatctcattataaaggccctgataatgataaacttctattaaaggattatatatatgaggctgaaatagctcttaacacttcagcaaatcgagcaatgaaaaatattgaaagatttatgattgaaagctctatttcatacttaaaaaataatctaattcccagtgtagtagaaaatttaaaaaaatttgatgctgatacaaaaaagaatttagatcaatttattgataaaaattcctcagtattaggatctgatttacatatattaaagtctcaagtagatttagaacttaatccaactactaaggtagcctttaatattcaaagtattccagattttgatataaatgcattgatagacagattaggtattcaattaaaagataacttagtatttagtttaggagtggaatctgataaaataaaagatctatctgggaataatacaaacctagaagttaaaacaggtgtccaaatagtagatggacgagatagtaagactatacgtttaaattcaaatgaaaattcaagtattatagttcagaaaaatgaaagtataaacttctcatattttagtgactttaccataagtttttggataagagttccaagacttaataaaaatgattttatagacttaggaattgaatatgacttagtaaataatatggataatcaaggatggaaaatttcgcttaaggatgggaatttagtatggagaatgaaagatagatttggaaaaataatagatattattacgtctttaacctttagtaatagctttatagataaatatatatccagtaatatatggagacatataactattacagttaaccaattaaaagattgtactttatatataaatggagataaaatagatagtaaatcaattaacgaattaagaggtatcgataataattctccaataatattcaagttagaagggaatagaaataaaaatcaatttatacgcttagatcagtttaatatttatcaaagggctttaaatgaaagtgaagttgaaatgttatttaatagttattttaattcaaatatattaagagatttttggggagaacctttagagtataataagagttactatatgataaatcaagcaatattaggtggaccccttagaagcacatataagtcatggtatggagagtattacccttatatatctagaatgaggacgtttaatgtttcatcatttattttaattccttacctatatcataaaggatcagatgtagaaaaggtaaaaataataaataaaaacaacgtggataaatatgtaagaaaaaatgatgtagcagatgttaaatttgaaaattatggtaatttaatacttacgttacctatgtacagtaaaatcaaagagagatatatggtattaaacgagggtagaaacggcgatttaaagttaattcaattacaaagtaacgataaatactattgtcaaatacgaatatttgaaatgtacagaaatgggttgctgtcaattgcagacgatgaaaactggttatactctagtggctggtatttatactctagtggctggtatttagataattataaaactttggatttaaaaaaacatacaaaaactaattggtattttgttagtgaagatgaaggatggaaggaatag

SEQ ID NO: 3 - protein, 1167 aa, Clostridium bifermentans
MDIIDNVDITLPENGEDIVIVGGRRYDYNGDLAKFKAFKVAKHIWVVPGRYYGEKLDIQDGEKINGGIYDKDFLSQNQEKQEFMDGVILLLKRINNTLEGKRLLSLITSAVPFPNEDDGIYKQNNFILSDKTFKAYTSNIIIFGPGANLVENKVIAFNSGDAENGLGTISEICFQPLLTYKFGDYFQDPALDLLKCLIKSLYYLYGIKVPEDFTLPYRLTNNPDKTEYSQVNMEDLLISGGDDLNAAGQRPYWLWNNYFIDAKDKFDKYKEIYENQMKLDPNLEINLSNHLEQKFNINISELWSLNISNFARTFNLKSPRSFYKALKYYYRKKYYKIHYNEIFGTNYNIYGFIDGQVNASLKETDLNIINKPQQIINLIDNNNILLIKSYIYDDELNKIDYNFYNNYEIPYNYGNSFKIPNITGILLPSVNYELIDKIPKIAEIKPYIKDSTPLPDSEKTPIPKELNVGIPLPIHYLDSQIYKGDEDKDFILSPDFLKVVSTKDKSLVYSFLPNIVSYFDGYDKTKISTDKKYYLWIREVLNNYSIDITRTENIIGIFGVDEIVPWMGRALNILNTENTFETELRKNGLKALLSKDLNVIFPKTKVDPIPTDNPPLTIEKIDEKLSDIYIKNKFFLIKNYYITIQQWWICCYSQFLNLSYMCREAIINQQNLIEKIILNQLSYLARETSINIETLYILSVTTEKTIEDLREISQKSMNNICNFFERASVSIFHTDIYNKFIDHMKYIVDDANTKIINYINSNSNITQEEKNYLINKYMLTEEDFNFFNFDKLINLFNSKIQLTIKNEKPEYNLLLSINQNESNENITDISGNNVKISYSNNINILDGRNEQAIYLDNDSQYVDFKSKNFENGVTNNFTISFWMRTLEKVDTNSTLLTSKLNENSAGWQLDLRRNGLVWSMKDHNKNEINIYLNDFLDISWHYIVVSVNRLTNILTVYIDGELSVNRNIEEIYNLYSDVGTIKLQASGSKVRIESFSILNRDIQRDEVSNRYINYIDNVNLRNIYGERLEYNKEYEVSNYVYPRNLLYKVNDIYLAIERGSNSSNRFKLILININEDKKFVQQKDIVIIKDVTQNKYLGISEDSNKIKLVDRNNALELILDNHLLNPNYTTFSTKQEEYLRLSNIDGIYNWVIKDVSRLNDIYSWTLI

SEQ ID NO: 4 - DNA, 3504 bp, Clostridium bifermentans
atggacataattgacaatgtagatataacattacctgaaaatggtgaagatattgtaatcgtaggaggaagaagatatgattataatggagacttagcaaaatttaaagcttttaaagtggctaagcatatttgggtggttccaggtagatattatggtgaaaaattagatatacaagatggtgaaaaaattaatggaggaatttatgacaaagattttttatctcagaatcaagaaaaacaagaatttatggatggagttatactcttattaaaaagaatcaataatacgttagaaggaaaaagattattatcgcttataacatccgctgtaccttttcctaacgaagatgatggaatatataaacaaaataactttatactttctgataaaacgtttaaagcgtatacttcaaatattattatttttggtcctggagcaaacttggtagagaataaagttattgcatttaatagtggtgatgctgaaaatggacttggaacaatatcagaaatttgttttcaaccgcttttaacttataaatttggagattattttcaggaccctgcactagatttattaaagtgtttaataaaatccttatattatttgtatggaattaaagttccagaagattttactttaccgtataggttgacgaataatccagataagacagaatattctcaggtcaatatggaagatttattaatatcaggtggtgatgatcttaatgctgcagggcagagaccatattggctatggaataattattttatagacgcaaaggataaatttgataaatataaagaaatttacgaaaaccaaatgaaactggatcctaatctagaaattaatctttcaaatcatttagagcaaaaatttaatataaacatatctgaattatggagcttaaacatatctaattttgcaagaacatttaatttaaaatcacctagaagtttttataaagcacttaaatattattatagaaaaaaatattataagatacattataatgaaatatttggaacaaattataatatatatggatttatagatggacaagttaatgcatcactaaaagaaactgatttaaatattataaataaaccacagcagattattaaccttattgataataacaatatattattaataaagtcctatatatatgacgatgaattaaataaaatagattataatttttataataattatgaaatcccttataactatggaaattcttttaaaatacctaatataacgggaatacttttacctagcgtaaattatgaattaattgataaaataccaaaaattgctgaaattaaaccttatattaaagactcaacaccattaccagattctgaaaaaacgcctattcctaaagagttaaatgtaggaattccattacctattcattatttggattcacaaatttataaaggagatgaagataaagattttatattatctcctgactttctaaaggttgtgtccaccaaagataaatctctagtatatagctttttacccaatattgtttcatattttgatggatatgataaaacaaaaatttctactgacaaaaaatattatttatggataagggaagttttaaataattattcaatagatataactagaactgaaaatataattggtatttttggagtagatgagatagttccttggatgggaagggccttgaatatcttaaatacagaaaatacttttgaaactgaacttagaaaaaatggcttaaaagctttgctttctaaagatttaaacgttattttcccaaaaacaaaagtggatccaatacctacagataatcctccccttacaatagaaaaaatagatgaaaaactttcagatatttatattaaaaataaattctttttaataaaaaattactacataactatacagcaatggtggatatgttgctatagtcaatttttaaatcttagttatatgtgtcgtgaagcaataataaatcaacaaaatttaattgaaaaaattattttaaatcaactcagctatttagctcgtgagacaagcattaacatagaaacgttgtatatattaagtgtaacaactgaaaagacaatagaagatttaagagaaatatcacaaaagtcaatgaataatatatgcaatttttttgaacgagctagtgtttcaatattccatactgatatttacaataagtttattgatcatatgaaatatatagttgatgatgcaaatactaagattataaattatataaattctaattctaatattacacaagaagaaaaaaattacttaattaataaatatatgctaacagaagaagattttaattttttcaattttgataaattaataaatttatttaattctaaaattcaactcacaattaaaaatgaaaagccggaatataatttattactatctataaatcaaaatgagagtaatgagaatattaccgatatatcaggaaataatgtaaaaattagttattcaaataatattaacatattagatggcagaaatgaacaggcaatatatttagataatgatagtcaatatgttgacttcaaatctaaaaattttgaaaatggagtaactaataattttacaattagtttttggatgagaactttagagaaagtagacacaaattctacattgttaacatctaaacttaatgagaattctgcaggatggcaactggatttaagaagaaatggattagtttggagtatgaaagatcacaacaaaaatgaaataaatatttatttaaatgattttttagatataagttggcactatatcgttgtttcagttaatcgtttaacaaatatattaactgtatatatagatggtgagcttagtgttaacagaaatattgaggaaatatataatctatattcagatgtggggacaattaaactgcaagcaagtggatctaaagttcgcattgaatctttttcgattttaaacagagacattcaaagagatgaggtatctaatagatacattaattatattgataatgtaaatttaaggaatatatatggggagagattagaatacaacaaggaatatgaagtatctaattatgtttatcctagaaacttactatacaaggtcaatgatatatatttagctattgagagaggaagcaacagttctaacaggtttaaattaatattaataaatataaatgaagataaaaaatttgtacagcaaaaagacatagttattattaaagatgtcactcaaaataaatatttaggtatttcagaagatagtaataagattaagctagtagatagaaataatgctttagagttgattctagataatcatcttcttaatcctaattatacgacattttctactaaacaagaagaatatttaagactatctaatatagatggaatatataactgggtgataaaggatgtatcgagattaaatgatatatattcttggactttaatataa

SEQ ID NO: 5 - protein, 142 aa, Clostridium bifermentans
MNREFPFHFNDGNVSMNGLFCLKKIKTQYHPNYDYFKIKFCEGFLSIKNKVKDDLCEYDLKNIESVIALKREYSKENNLKNKESAIFMNIGNKGIHNKYDLYVVNVDINNILDENYMLKGILNDKLKILFLGNERKLLRIKN

SEQ ID NO: 6 - DNA, 429 bp, Clostridium bifermentans
atgaatagggagtttccattccattttaatgatgggaatgtttcgatgaatggattattttgtttaaagaaaataaaaacgcaatatcatccaaattatgattatttcaaaattaaattctgtgaagggtttttatctataaagaataaggttaaagatgatttgtgtgaatatgatttgaaaaacattgaatccgtaattgcattaaaaagagaatattcaaaagaaaataatttaaaaaataaagaatcagcaatttttatgaatattgggaataaagggattcataataaatatgatttatatgttgtaaatgtagatattaacaatattttagatgaaaattatatgttaaaaggaatattaaatgataagctaaagattctttttttaggtaatgaaaggaagttattaagaataaaaaattag

SEQ ID NO: 7 - protein, 740 aa, Clostridium bifermentans
MSKKPLDFLRIYDWHKTEAMNKISKLDFERIIPKHFSKEIKNKHLSVKITGNWKIWKLTDEGEGQYPIFKCIVEDGFLKIKNECGNKKYSLDNAWIKICTKIKYDNENGKDIYSIDEKNLTLYSVNNSFNSKYKNNIVDAFLDNLLIACIEDNIKDLNKFFKLYKVKTAIKEDLSLLGWDTGYSTSFTHVNKTIENQQNYPKQFKYESEGPYNIDISGEFDSWRLTTGSDGQNVNFICPIKNGEFNFLGTEYKFSQGEQVNIQLKLKYLNIEEPTFEDSTSLNDGNQVDLIVKTDEDENENPPVTIIKVVLLGEIDAIGKMLLEGTFREWFNENIDAFKQIFSSFLLEDTSKNPDFQWLKPTKAYYGVASAEPIDGKPDLDSSVFSVMSMVEDNKNDKPSHTVDGRILDAVNNESAFGIRTPLFVKKWLIAGLEMMQIGKLEDFDLINNGMGFINNKKLLFGTFENADGEDVPAYVEKDNFRLEITNNQLKIEITDIYWQQSRRLTGHVMYSQYFDLELRSGTDITGAEYKNILIPVENSEPTLVVNISQDEFDIWGDIVGEIVGGIVVGIVTGYLGSILGKGVGKYLEKFLTKTSGGRWVLKMNKEMYDYLNNLFKGDRRVFNEVAIDEIELISTLGTSQAISTIANTPTNFASKIWVNKSKFIGGLIGGSVGSVIPSVIIKSIDAWDKQNYSVLPSINAFVASSVGSVKWPDTSEFKIESAELNGIFLLGGKLERYEK

SEQ ID NO: 8 - DNA, 2223 bp, Clostridium bifermentans
atgagtaaaaaaccattagattttctaagaatttatgattggcataaaactgaagcaatgaacaaaattagtaaactagattttgaaaggataattcctaaacatttttcaaaagaaattaaaaataaacacttaagtgttaaaattactggtaactggaaaatttggaagttaacagatgaaggagaagggcaatatcctatttttaaatgcatagttgaagatggattcttaaaaataaaaaatgaatgtggaaataaaaaatattcactagataatgcttggataaaaatttgtacaaaaattaaatatgataatgaaaatggaaaagatatctattcaatagatgaaaaaaacttaacattgtacagtgttaataattcatttaactcaaaatataaaaataatattgtagatgcttttttagataatttattaatagcgtgtattgaggacaatataaaagatttaaataagttttttaagctatataaagttaaaacagcaataaaagaagatttaagtctcttaggatgggatacaggatactcaacatcatttactcatgtaaataaaactattgaaaatcaacagaattatccgaagcagtttaaatatgagtctgagggtccttataacattgatatatctggagaatttgattcatggagattaactactggatcagatggtcaaaatgttaattttatttgtccaattaaaaatggtgaatttaactttttgggaaccgagtataaattttcacaaggtgaacaagttaatatacaacttaagttaaaatatttaaatattgaagagccaacctttgaagattcaacttccttaaatgatggaaatcaggttgatttaattgttaaaacagatgaagacgagaatgaaaatcctccggttacaattataaaagtagttttactaggtgaaattgacgctattggtaagatgcttttagagggtacgtttagagagtggtttaatgaaaatattgatgcatttaaacaaatattttcttctttccttttagaggatacatctaaaaatccagattttcagtggttaaaacctacaaaggcttattatggagttgcaagtgctgaaccaatagacggaaagcctgacttagatagtagtgtattttctgtcatgtctatggtagaagataataaaaatgataaaccaagtcatacagtagatggtagaatacttgatgctgttaataatgaatctgcatttggaattagaaccccattatttgttaaaaaatggcttattgccggactagaaatgatgcaaattggaaaattagaagattttgatttaataaataacggaatgggatttattaataacaagaaacttttgtttggtacttttgaaaatgctgatggtgaagatgtacctgcttatgtagaaaaagataattttagattagaaataacgaataatcaactaaaaatagaaataacagatatatattggcagcaatcaagaagattaacagggcatgtaatgtatagccaatattttgatttagaattaagaagcggaactgatatcactggagcagaatataaaaatattttaattccagtagaaaattcagagccaacattggtagtaaacatttcacaagatgaatttgatatttggggagatattgtcggtgaaatagttggaggtatagttgtgggaatagtcacaggttacttaggtagcattttaggcaaaggagtaggaaaatatttagaaaaattccttacaaaaacatctggtggaagatgggtattaaaaatgaataaagagatgtatgattatttaaataatttatttaaaggagatagaagagttttcaatgaagttgccatagatgaaatagaactgatttcaacattaggaacatctcaagctatatcaacaattgcaaatacacctactaattttgcatctaaaatatgggtaaataaatcaaaatttataggtggtttaattggggggtcagtaggctcagtaatacctagcgttattataaaatcaatagacgcttgggataaacaaaattattctgttcttccaagtataaatgcatttgtagcttcaagtgtaggttctgtaaaatggccggataccagtgaattcaagattgaatcagctgagcttaacggaatttttttgttaggtggaaagctagaaagatatgaaaaataa

SEQ ID NO: 9 - protein, 491 aa, Clostridium bifermentans
MIGKRQTSTLNWDTVFAVPISVVNKAIKDKKSSPENFEFEDSSGSKCKGDFGDWQIITGGDGSNIRMKIPIYNFKAELVDDKYGIFNGNGGFESGEMNIQVKLKYFPHDKISKYKDVELVDLKVRSESADPIDPVVVMLSLKNLNGFYFNFLNEFGEDLQDIIEMFFIELVKQWLTENISLFNHIFSVVNLNLYIDQYSQWSWSRPSYVSYAYTDIEGDLDKSLLGVLCMTGGRNPDLRQQKVDPHAVPESSQCGFLIYEERVLRDLLLPTLPMKFKNSTVEDYEVINASGESGQYQYILRLKKGRSVSLDRVEANGSKYDPYMTEMSISLSNDVLKLEATTETSVGMGGKVGCDTINWYKLVLAKNGNGEQTISYEEVGEPTVINYVIKEGENWVWDVIAAIIAILATAVLAIFTGGAAFFIGGIVIAIITGFIAKTPDIILNWNLETSPSIDMMLENSTSQIIWNARDIFELDYVALNGPLQLGGELTV

SEQ ID NO: 10 - DNA, 1476 bp, Clostridium bifermentans
atgataggaaaacgtcaaacaagtacactgaattgggatacagtatttgctgttcctattagtgtagtaaataaagcgataaaagataaaaaaagtagccctgagaattttgaatttgaagattcatctggtagtaaatgtaaaggggattttggagattggcaaataattactggtggtgatggaagtaatatacgaatgaaaattcctatttacaattttaaagctgaactggtcgatgataaatatggaatttttaatggaaacggtggatttgaatctggagaaatgaatattcaagttaagcttaagtattttccacatgataaaatatcaaaatataaagatgttgaattagttgatttaaaagtaagatcagaaagtgctgatccaattgatccagtagtagttatgctctcattgaagaatttaaatgggttttattttaattttttaaatgaatttggtgaagatttacaagatattatagagatgttttttatagagctcgttaaacaatggctgacagaaaatattagtttatttaaccatatttttagtgtagtaaacttaaatttatatattgatcaatattctcaatggtcatggagtaggccttcatatgttagctatgcttatacagatatagaaggtgatttagataaaagtctattaggggttttgtgtatgacaggaggaagaaatcctgatcttagacaacagaaggtagatcctcatgcagtaccagaaagttctcaatgtggatttttaatttatgaagagagggtattaagagatttacttttaccaactttaccaatgaaatttaaaaattcaacagtagaagattatgaggtaattaatgcaagcggagaaagtggtcagtatcagtatatattaagattaaaaaaaggtaggagtgttagtttagaccgcgttgaggctaatggttctaaatatgatccatatatgactgaaatgagtattagtttatcaaatgatgtattaaaactagaagcaaccacagaaacttcggtaggaatgggaggaaaagttggatgtgatactataaattggtataagttagtacttgcaaaaaatggaaatggagaacaaactatatcatatgaagaagttggagaacctacagtaataaattatgtaataaaagaaggcgaaaattgggtatgggatgtaatcgctgcaatcatagctattctagcaacagcagtattggcaatatttactggaggagcagctttttttataggtggtattgttatagctataataacaggatttatagctaaaactccagatataattttaaattggaaccttgaaacttctccaagtatagatatgatgttagaaaattctacttcacaaattatttggaatgctagagacatatttgaactagattatgttgctttaaatggaccactgcaactaggtggagaattaactgtttaa

SEQ ID NO: 11 - DNA, 11675 bp, Clostridium bifermentans
atggacataattgacaatgtagatataacattacctgaaaatggtgaagatattgtaatcgtaggaggaagaagatatgattataatggagacttagcaaaatttaaagcttttaaagtggctaagcatatttgggtggttccaggtagatattatggtgaaaaattagatatacaagatggtgaaaaaattaatggaggaatttatgacaaagattttttatctcagaatcaagaaaaacaagaatttatggatggagttatactcttattaaaaagaatcaataatacgttagaaggaaaaagattattatcgcttataacatccgctgtaccttttcctaacgaagatgatggaatatataaacaaaataactttatactttctgataaaacgtttaaagcgtatacttcaaatattattatttttggtcctggagcaaacttggtagagaataaagttattgcatttaatagtggtgatgctgaaaatggacttggaacaatatcagaaatttgttttcaaccgcttttaacttataaatttggagattattttcaggaccctgcactagatttattaaagtgtttaataaaatccttatattatttgtatggaattaaagttccagaagattttactttaccgtataggttgacgaataatccagataagacagaatattctcaggtcaatatggaagatttattaatatcaggtggtgatgatcttaatgctgcagggcagagaccatattggctatggaataattattttatagacgcaaaggataaatttgataaatataaagaaatttacgaaaaccaaatgaaactggatcctaatctagaaattaatctttcaaatcatttagagcaaaaatttaatataaacatatctgaattatggagcttaaacatatctaattttgcaagaacatttaatttaaaatcacctagaagtttttataaagcacttaaatattattatagaaaaaaatattataagatacattataatgaaatatttggaacaaattataatatatatggatttatagatggacaagttaatgcatcactaaaagaaactgatttaaatattataaataaaccacagcagattattaaccttattgataataacaatatattattaataaagtcctatatatatgacgatgaattaaataaaatagattataatttttataataattatgaaatcccttataactatggaaattcttttaaaatacctaatataacgggaatacttttacctagcgtaaattatgaattaattgataaaataccaaaaattgctgaaattaaaccttatattaaagactcaacaccattaccagattctgaaaaaacgcctattcctaaagagttaaatgtaggaattccattacctattcattatttggattcacaaatttataaaggagatgaagataaagattttatattatctcctgactttctaaaggttgtgtccaccaaagataaatctctagtatatagctttttacccaatattgtttcatattttgatggatatgataaaacaaaaatttctactgacaaaaaatattatttatggataagggaagttttaaataattattcaatagatataactagaactgaaaatataattggtatttttggagtagatgagatagttccttggatgggaagggccttgaatatcttaaatacagaaaatacttttgaaactgaacttagaaaaaatggcttaaaagctttgctttctaaagatttaaacgttattttcccaaaaacaaaagtggatccaatacctacagataatcctccccttacaatagaaaaaatagatgaaaaactttcagatatttatattaaaaataaattctttttaataaaaaattactacataactatacagcaatggtggatatgttgctatagtcaatttttaaatcttagttatatgtgtcgtgaagcaataataaatcaacaaaatttaattgaaaaaattattttaaatcaactcagctatttagctcgtgagacaagcattaacatagaaacgttgtatatattaagtgtaacaactgaaaagacaatagaagatttaagagaaatatcacaaaagtcaatgaataatatatgcaatttttttgaacgagctagtgtttcaatattccatactgatatttacaataagtttattgatcatatgaaatatatagttgatgatgcaaatactaagattataaattatataaattctaattctaatattacacaagaagaaaaaaattacttaattaataaatatatgctaacagaagaagattttaattttttcaattttgataaattaataaatttatttaattctaaaattcaactcacaattaaaaatgaaaagccggaatataatttattactatctataaatcaaaatgagagtaatgagaatattaccgatatatcaggaaataatgtaaaaattagttattcaaataatattaacatattagatggcagaaatgaacaggcaatatatttagataatgatagtcaatatgttgacttcaaatctaaaaattttgaaaatggagtaactaataattttacaattagtttttggatgagaactttagagaaagtagacacaaattctacattgttaacatctaaacttaatgagaattctgcaggatggcaactggatttaagaagaaatggattagtttggagtatgaaagatcacaacaaaaatgaaataaatatttatttaaatgattttttagatataagttggcactatatcgttgtttcagttaatcgtttaacaaatatattaactgtatatatagatggtgagcttagtgttaacagaaatattgaggaaatatataatctatattcagatgtggggacaattaaactgcaagcaagtggatctaaagttcgcattgaatctttttcgattttaaacagagacattcaaagagatgaggtatctaatagatacattaattatattgataatgtaaatttaaggaatatatatggggagagattagaatacaacaaggaatatgaagtatctaattatgtttatcctagaaacttactatacaaggtcaatgatatatatttagctattgagagaggaagcaacagttctaacaggtttaaattaatattaataaatataaatgaagataaaaaatttgtacagcaaaaagacatagttattattaaagatgtcactcaaaataaatatttaggtatttcagaagatagtaataagattaagctagtagatagaaataatgctttagagttgattctagataatcatcttcttaatcctaattatacgacattttctactaaacaagaagaatatttaagactatctaatatagatggaatatataactgggtgataaaggatgtatcgagattaaatgatatatattcttggactttaatataaactattaaaaattttaaaataaggaggttgtatcaacttcaaatgcatgctaatcaatgtttaatacattagaaattagaaggggggggtaagatgaatagggagtttccattccattttaatgatgggaatgtttcgatgaatggattattttgtttaaagaaaataaaaacgcaatatcatccaaattatgattatttcaaaattaaattctgtgaagggtttttatctataaagaataaggttaaagatgatttgtgtgaatatgatttgaaaaacattgaatccgtaattgcattaaaaagagaatattcaaaagaaaataatttaaaaaataaagaatcagcaatttttatgaatattgggaataaagggattcataataaatatgatttatatgttgtaaatgtagatattaacaatattttagatgaaaattatatgttaaaaggaatattaaatgataagctaaagattctttttttaggtaatgaaaggaagttattaagaataaaaaattagggggaggaattatgagtaaaaaaccattagattttctaagaatttatgattggcataaaactgaagcaatgaacaaaattagtaaactagattttgaaaggataattcctaaacatttttcaaaagaaattaaaaataaacacttaagtgttaaaattactggtaactggaaaatttggaagttaacagatgaaggagaagggcaatatcctatttttaaatgcatagttgaagatggattcttaaaaataaaaaatgaatgtggaaataaaaaatattcactagataatgcttggataaaaatttgtacaaaaattaaatatgataatgaaaatggaaaagatatctattcaatagatgaaaaaaacttaacattgtacagtgttaataattcatttaactcaaaatataaaaataatattgtagatgcttttttagataatttattaatagcgtgtattgaggacaatataaaagatttaaataagttttttaagctatataaagttaaaacagcaataaaagaagatttaagtctcttaggatgggatacaggatactcaacatcatttactcatgtaaataaaactattgaaaatcaacagaattatccgaagcagtttaaatatgagtctgagggtccttataacattgatatatctggagaatttgattcatggagattaactactggatcagatggtcaaaatgttaattttatttgtccaattaaaaatggtgaatttaactttttgggaaccgagtataaattttcacaaggtgaacaagttaatatacaacttaagttaaaatatttaaatattgaagagccaacctttgaagattcaacttccttaaatgatggaaatcaggttgatttaattgttaaaacagatgaagacgagaatgaaaatcctccggttacaattataaaagtagttttactaggtgaaattgacgctattggtaagatgcttttagagggtacgtttagagagtggtttaatgaaaatattgatgcatttaaacaaatattttcttctttccttttagaggatacatctaaaaatccagattttcagtggttaaaacctacaaaggcttattatggagttgcaagtgctgaaccaatagacggaaagcctgacttagatagtagtgtattttctgtcatgtctatggtagaagataataaaaatgataaaccaagtcatacagtagatggtagaatacttgatgctgttaataatgaatctgcatttggaattagaaccccattatttgttaaaaaatggcttattgccggactagaaatgatgcaaattggaaaattagaagattttgatttaataaataacggaatgggatttattaataacaagaaacttttgtttggtacttttgaaaatgctgatggtgaagatgtacctgcttatgtagaaaaagataattttagattagaaataacgaataatcaactaaaaatagaaataacagatatatattggcagcaatcaagaagattaacagggcatgtaatgtatagccaatattttgatttagaattaagaagcggaactgatatcactggagcagaatataaaaatattttaattccagtagaaaattcagagccaacattggtagtaaacatttcacaagatgaatttgatatttggggagatattgtcggtgaaatagttggaggtatagttgtgggaatagtcacaggttacttaggtagcattttaggcaaaggagtaggaaaatatttagaaaaattccttacaaaaacatctggtggaagatgggtattaaaaatgaataaagagatgtatgattatttaaataatttatttaaaggagatagaagagttttcaatgaagttgccatagatgaaatagaactgatttcaacattaggaacatctcaagctatatcaacaattgcaaatacacctactaattttgcatctaaaatatgggtaaataaatcaaaatttataggtggtttaattggggggtcagtaggctcagtaatacctagcgttattataaaatcaatagacgcttgggataaacaaaattattctgttcttccaagtataaatgcatttgtagcttcaagtgtaggttctgtaaaatggccggataccagtgaattcaagattgaatcagctgagcttaacggaatttttttgttaggtggaaagctagaaagatatgaaaaataatagaataaaaggataataataaaaagataagatagaaaaatttgtcttatctttttataaatatagtttgaaaggggaatttaaactatgataggaaaacgtcaaacaagtacactgaattgggatacagtatttgctgttcctattagtgtagtaaataaagcgataaaagataaaaaaagtagccctgagaattttgaatttgaagattcatctggtagtaaatgtaaaggggattttggagattggcaaataattactggtggtgatggaagtaatatacgaatgaaaattcctatttacaattttaaagctgaactggtcgatgataaatatggaatttttaatggaaacggtggatttgaatctggagaaatgaatattcaagttaagcttaagtattttccacatgataaaatatcaaaatataaagatgttgaattagttgatttaaaagtaagatcagaaagtgctgatccaattgatccagtagtagttatgctctcattgaagaatttaaatgggttttattttaattttttaaatgaatttggtgaagatttacaagatattatagagatgttttttatagagctcgttaaacaatggctgacagaaaatattagtttatttaaccatatttttagtgtagtaaacttaaatttatatattgatcaatattctcaatggtcatggagtaggccttcatatgttagctatgcttatacagatatagaaggtgatttagataaaagtctattaggggttttgtgtatgacaggaggaagaaatcctgatcttagacaacagaaggtagatcctcatgcagtaccagaaagttctcaatgtggatttttaatttatgaagagagggtattaagagatttacttttaccaactttaccaatgaaatttaaaaattcaacagtagaagattatgaggtaattaatgcaagcggagaaagtggtcagtatcagtatatattaagattaaaaaaaggtaggagtgttagtttagaccgcgttgaggctaatggttctaaatatgatccatatatgactgaaatgagtattagtttatcaaatgatgtattaaaactagaagcaaccacagaaacttcggtaggaatgggaggaaaagttggatgtgatactataaattggtataagttagtacttgcaaaaaatggaaatggagaacaaactatatcatatgaagaagttggagaacctacagtaataaattatgtaataaaagaaggcgaaaattgggtatgggatgtaatcgctgcaatcatagctattctagcaacagcagtattggcaatatttactggaggagcagctttttttataggtggtattgttatagctataataacaggatttatagctaaaactccagatataattttaaattggaaccttgaaacttctccaagtatagatatgatgttagaaaattctacttcacaaattatttggaatgctagagacatatttgaactagattatgttgctttaaatggaccactgcaactaggtggagaattaactgtttaaaattaaaaattttaataagaataatttttatatatttattatagataccttaaaggagtagggaaatgtatgctacaaataagagtttttaattataatgatccaattgatggagaaaatatcgtggagttaagataccataacaggagccctgtaaaagcatttcaaatagtagatggtatatggataattccagaaagatataactttacaaacgatacaaaaaaagttccagacgatcgagctcttactattctggaagatgaagtttttgctgttcgcgaaaatgactatttaacaacagatgttaatgaaaaaaattcctttttaaataatattactaagctttttaagcgtattaattcaagtaacattggtaatcagttacttaattatatttcaacaagcgtcccatatccagttgtgagtacaaattcaataaaggctagagactataatacaattaaatttgattcaattgatgggcgaagaattacaaaatctgcaaatgtacttatctacggaccaagtatgaaaaatttactagataaacaaacaagggctatcaatggggaagaagcaaaaaatggtataggatgtttaagtgatattattttttctccaaattacttatctgtccaaactgtttcttcaagtaggtttgttgaagatcctgcatcatcacttacacatgaacttatccatgccttacataatttatatggaatacaatatcctggagaagaaaaatttaaatttggaggatttattgataaactattaggaactagagaatgcatagattatgaggaagtcttaacatatggaggaaaagattccgaaattataagaaagaaaattgataagtccttatatcctgatgattttgtaaataagtatggtgaaatgtataagcgtataaaaggatctaatccttattatcccgacgaaaaaaaattaaaacaaagttttttaaacagaatgaatccatttgatcaaaatggaacttttgatactaaagaatttaaaaatcatcttatggatttatggtttgggttaaatgagagtgaatttgctaaagaaaagaagattttagtcagaaagcactatataacaaagcaaattaatcctaaatacacagaacttactaatgatgtatatactgaagataaaggctttgtaaatggtcaatctatagacaatcaaaattttaaaataattgatgatttaatatcaaaaaaagtaaaactatgttctataacatctaaaaatcgagtaaatatttgtatagacgttaataaagaagatttatatttcataagtgataaagaaggttttgaaaatatagatttttccgagccggaaattagatatgatagtaatgtaactacagcaactacctcttcttttacagaccattttttagtaaatagaacttttaacgatagtgatagatttccacctgtagaattagaatatgctatcgaaccagctgaaatagttgataacactataatgccagatattgatcaaaaaagcgaaatatctctcgataacttaacgacctttcactatttaaatgctcaaaaaatggatttgggatttgattcatcaaaagaacagttaaagatggttacatcaatagaggaatcattattagattcaaaaaaggtatacacaccatttacgagaactgcacatagtgtaaatgaacgtatatctggaatagcggaaagttacttattttatcaatggttaaaaactgttataaatgattttacagatgaattaaaccaaaagagtaatactgacaaagttgctgatatttcttggattataccctatgttggacctgctttaaatattggccttgatttatctcatggagattttactaaagcttttgaagatttaggggtttctattttatttgctattgctccagaatttgcaactataagtcttgtagctctttcaatatatgaaaatatagaagaggattcacaaaaagaaaaagtaattaataaagtagaaaatacattagcaaggagaatagaaaaatggcaccaagtttatgctttcatggtggctcagtggtggggtatggttcatactcagatagacactagaattcatcaaatgtatgaatcactttctcatcaaattatagcaattaaagctaatatggagtatcagttatctcattataaaggccctgataatgataaacttctattaaaggattatatatatgaggctgaaatagctcttaacacttcagcaaatcgagcaatgaaaaatattgaaagatttatgattgaaagctctatttcatacttaaaaaataatctaattcccagtgtagtagaaaatttaaaaaaatttgatgctgatacaaaaaagaatttagatcaatttattgataaaaattcctcagtattaggatctgatttacatatattaaagtctcaagtagatttagaacttaatccaactactaaggtagcctttaatattcaaagtattccagattttgatataaatgcattgatagacagattaggtattcaattaaaagataacttagtatttagtttaggagtggaatctgataaaataaaagatctatctgggaataatacaaacctagaagttaaaacaggtgtccaaatagtagatggacgagatagtaagactatacgtttaaattcaaatgaaaattcaagtattatagttcagaaaaatgaaagtataaacttctcatattttagtgactttaccataagtttttggataagagttccaagacttaataaaaatgattttatagacttaggaattgaatatgacttagtaaataatatggataatcaaggatggaaaatttcgcttaaggatgggaatttagtatggagaatgaaagatagatttggaaaaataatagatattattacgtctttaacctttagtaatagctttatagataaatatatatccagtaatatatggagacatataactattacagttaaccaattaaaagattgtactttatatataaatggagataaaatagatagtaaatcaattaacgaattaagaggtatcgataataattctccaataatattcaagttagaagggaatagaaataaaaatcaatttatacgcttagatcagtttaatatttatcaaagggctttaaatgaaagtgaagttgaaatgttatttaatagttattttaattcaaatatattaagagatttttggggagaacctttagagtataataagagttactatatgataaatcaagcaatattaggtggaccccttagaagcacatataagtcatggtatggagagtattacccttatatatctagaatgaggacgtttaatgtttcatcatttattttaattccttacctatatcataaaggatcagatgtagaaaaggtaaaaataataaataaaaacaacgtggataaatatgtaagaaaaaatgatgtagcagatgttaaatttgaaaattatggtaatttaatacttacgttacctatgtacagtaaaatcaaagagagatatatggtattaaacgagggtagaaacggcgatttaaagttaattcaattacaaagtaacgataaatactattgtcaaatacgaatatttgaaatgtacagaaatgggttgctgtcaattgcagacgatgaaaactggttatactctagtggctggtatttatactctagtggctggtatttagataattataaaactttggatttaaaaaaacatacaaaaactaattggtattttgttagtgaagatgaaggatggaaggaatag

SEQ ID NO: 12 - DNA, 30 bp, artificial
  Primer 1
ggcgcgccatggacataattgacaatgtag

SEQ ID NO: 13 - DNA, 27 bp, Artificial Sequence
  Primer 2
ctcgagctattccttccatccttcatc

SEQ ID NO: 14 - DNA, 31 bp, Artificial Sequence
  Primer 3
cccgggatccaataatagaaggatatcaaat

SEQ ID NO: 15 - DNA, 33 bp, Artificial Sequence
  Primer 4
gcggccgcccattcatcgaaacattcccatcat

SEQ ID NO: 16 - DNA, 31 bp, Artificial Sequence
  Primer 5
ctcgagatatttattatagataccttaaagg

SEQ ID NO: 17 - DNA, 37 bp, Artificial Sequence
  Primer 6
ccacttaattggtcaaataactattcttaatatgcta

SEQ ID NO: 18 - DNA, 41 bp, Artificial Sequence
  Primer 7
cggcatcgagcctgacgcaccaactgatccatgctctgcac

SEQ ID NO: 19 - DNA, 41 bp, Artificial Sequence
  Primer 8
gtgcagagcatggatcagttggtgcgtcaggctcgatgccg

SEQ ID NO: 20 - DNA, 34 bp, Artificial Sequence
  Primer 9
ggatccctgcaaatccgtgtctttaactataacg

SEQ ID NO: 21 - DNA, 32 bp, Artificial Sequence
  Primer 10
gggcccacatacgggataatccaagagatgtc

SEQ ID NO: 22 - DNA, 32 bp, Artificial Sequence
  Primer 11
ggatccgaatgccctgatcgatcgcctgggta

SEQ ID NO: 23 - DNA, 31 bp, Artificial Sequence
  Primer 12
aagctttcattctttccaaccttcatcttcc

SEQ ID NO: 24 - DNA, 57 bp, Artificial Sequence
  Primer 13
ccatggactacaaagacgatgacgacaagctgcaaatccgtgtctttaactataacg

SEQ ID NO: 25 - DNA, 33 bp, Artificial Sequence
  Primer 14
aagctttcacagtttaacttttttcgagatcag

SEQ ID NO: 26 - DNA, 35 bp, Artificial Sequence
  Primer 15
cgggatccgatgacgaaggacagattagcagccct

SEQ ID NO: 27 - DNA, 27 bp, Artificial Sequence
  Primer 16
ggcgcgccttacaggtcttcttcagag

SEQ ID NO: 28 - DNA, 38 bp, Artificial Sequence
  Primer 17
gattgatcgtatagaatataacgtcgaacatgcaatgg

SEQ ID NO: 29 - DNA, 38 bp, Artificial Sequence
  Primer 18
ccattgcatgttcgacgttatattctatacgatcaatc

SEQ ID NO: 30 - DNA, 38 bp, Artificial Sequence
  Primer 19
caagacacaaagaaagcggtcaaatatcaaagcaaagc

SEQ ID NO: 31 - DNA, 38 bp, Artificial Sequence
  Primer 20
gctttgctttgatatttgaccgctttctttgtgtcttg

SEQ ID NO: 32 - DNA, 40 bp, Artificial Sequence
  Primer 21
gattatgttcaaacagcggtgtctgacacaaagaaagcgc

SEQ ID NO: 33 - DNA, 40 bp, Artificial Sequence
  Primer 22
gcgctttctttgtgtcagacaccgctgtttgaacataatc

SEQ ID NO: 34 - DNA, 38 bp, Artificial Sequence
  Primer 23
caatggattatgttgaaagagcgacacaagacacaaag

SEQ ID NO: 35 - DNA, 38 bp, Artificial Sequence
  Primer 24
ctttgtgtcttgtgtcgctctttcaacataatccattg

SEQ ID NO: 36 - DNA, 38 bp, Artificial Sequence
  Primer 25
cacgtcgaacatgcagtggattatgttcaaacagcgac

SEQ ID NO: 37 - DNA, 38 bp, Artificial Sequence
  Primer 26
gtcgctgtttgaacataatccactgcatgttcgacgtg

SEQ ID NO: 38 - DNA, 60 bp, Artificial Sequence
  Primer 27
gttccaggtcttcttcagagatcagtttctgttcgctttgatatttaagcgctttctttg

SEQ ID NO: 39 - protein, 15 aa, Clostridium bifermentans
HAMDYVQTATQDTKK

SEQ ID NO: 40 - protein, 13 aa, Clostridium bifermentans
ALKYQSEQKLISE

SEQ ID NO: 41 - protein, 11 aa, Clostridium bifermentans
LEQKLISEEDL

SEQ ID NO: 42 - protein, 14 aa, Clostridium bifermentans
GFENIDFSEPEIRY

SEQ ID NO: 43 - protein, 55 aa, Clostridium bifermentans
RNGLLSIADDENWLYSSGWYLYSSGWYLDNYKTLDLKKHTKTNWYFVSEDEGWKE

SEQ ID NO: 44 - protein, 58 aa, Clostridium botulinum
EIGLIGIHRFYESGIVFKEYKDYFCISKWYLKEVKRKPYNSKLGCNWQFIPKDEGWTE

SEQ ID NO: 45 - protein, 59 aa, Clostridium botulinum
TFGLFGIGKFVKDYGYVWDTYDNYFCISQWYLRRISENINKLRLGCNWQFIPVDEGWTE

SEQ ID NO: 46 - protein, 51 aa, Clostridium botulinum
DIGFIGFHQFNNIAKLVASNWYNRQIERSSRTLGCSWEFIPVDDGWGERPL

SEQ ID NO: 47 - protein, 45 aa, Clostridium botulinum
NIGLLGFKADTVVASTWYYTHMRDHTNSNGCFWNFISEEHGWQEK

SEQ ID NO: 48 - protein, 45 aa, Clostridium botulinum
NIGLLGFHSNNLVASSWYYNNIRKNTSSNGCFWSFISKEHGWQEN

SEQ ID NO: 49 - protein, 23 aa, Clostridium botulinum
FATDPAQVTLAHELIHAGHRLYG

SEQ ID NO: 50 - protein, 22 aa, Clostridium botulinum
FIQDPALTLMHELIHSLHGLYG

SEQ ID NO: 51 - protein, 22 aa, Clostridium tetani
YFQDPALLLMHELIHVLHGLYG

SEQ ID NO: 52 - protein, 22 aa, Clostridium botulinum
YFSDPALILMHELIHVLHGLYG

SEQ ID NO: 53 - protein, 22 aa, Clostridium botulinum
YFADPALTLMHELIHVLHGLYG

SEQ ID NO: 54 - protein, 22 aa, Clostridium bifermentans
FVEDPASSLTHELIHALHNLYG

SEQ ID NO: 55 - protein, 22 aa, Clostridium botulinum
FIADPAISLAHELIHALHGLYG

SEQ ID NO: 56 - protein, 22 aa, Clostridium botulinum
FCMDPILILMHELNHAMHNLYG

SEQ ID NO: 57 - protein, 22 aa, Clostridium botulinum
FCMDPVIALMHELTHSLHQLYG

SEQ ID NO: 58 - protein, 295 aa, Anopheles gambiae
MGSSHHHHHHSQDPMTKDRLAALQAAQSDDEDMPEDVAVPVEGSFMEDFFKEVEEIRMMIDKIQANVEEVKKKHSAILSAPQSDEKTKQELEDLMADIKKTANRVRGKLKGIEQNIEQEEQQSKSNADLRIRKTQHSALSRKFVEVMTEYNRTQTDYRERCKGRIQRQLEITGRATTNEELEEMLEQGNSAVFTQGIIMETQQAKQTLADIEARHADIIKLENSIRELHDMFMDMAMLVESQGEMIDRIEYHVEHAMDYVQTATQDTKKALKYQSEQKLISEEDLEQKLISEEDL

SEQ ID NO: 59 - protein, 38 aa, Anopheles gambiae
GEMDRIEYHVEHAMDYVQTATQDTKKALKYQSKARRKK

SEQ ID NO: 60 - protein, 39 aa, Drosophila melanogaster
GEMIDRIEYHVEHAMDYVQTATQDTKKALKYQSKARRKK

SEQ ID NO: 61 - protein, 39 aa, Tribolium castaneum
GEMIDRIEYHVEHAVDYVQTATQDTKKALKYQSKARRKK

SEQ ID NO: 62 - protein, 39 aa, Apis mellifera; identical to SEQ ID NO: 61

SEQ ID NO: 63 - protein, 39 aa, Daphnia pulex
GEMIDRIEYNVEHAVDYVQTATQDTKKALKYQSKARRKK

SEQ ID NO: 64 - protein, 39 aa, Danio rerio
GEMIDRIEYNVEHSVDYVERAVSDTKKAVKYQSQARKKK

SEQ ID NO: 65 - protein, 39 aa, Xenopus tropicalis
GEMIDRIEYNVEHSVDYVERAVSDTKKAVKYQSKARRKK

SEQ ID NO: 66 - protein, 38 aa, Gallus gallus
GEMIDRIEYNVEHSVDYVERAVSDTKAVKYQSKARRKK

SEQ ID NO: 67 - protein, 39 aa, Mus musculus
GEMIDRIEYNVEHAVDYVERAVSDTKKAVKYQSKARRKK

SEQ ID NO: 68 - protein, 39 aa, Homo sapiens; identical to SEQ ID NO: 67

(19) United States
(12) Patent Application Publication
(10) Pub. No.: US 2019/0364907 A1
(43) Pub. Date: Dec. 5, 2019

(54) COMPOSITIONS OF MOSQUITOCIDAL CLOSTRIDIAL PROTEINS AND METHODS OF USE

(71) Applicant: The Regents of the University of California, Oakland, CA (US)

(72) Inventors: Sarjeet S. Gill, Riverside, CA (US); Estefania Contreras Navarro, Riverside, CA (US); Jianwu Chen, Riverside, CA (US)

(21) Appl. No.: 16/041,703

(22) Filed: Jul. 20, 2018

Related U.S. Application Data
(60) Provisional application No. 62/535,746, filed on Jul. 21, 2017.

(51) Int. Cl. A01N 63/02; C07K 14/32; C12R 1/07

(52) U.S. Cl. CPC A01N 63/02; C12N 15/81; C12R 1/07; C07K 14/32

(57) ABSTRACT

Mosquitocidal compositions and methods include a microbe genetically modified to express a heterologous clostridial mosquitocidal protein 1 (CMP1) protein having an amino acid sequence of SEQ ID NO: 1 or a variant thereof and a heterologous non-toxic non-hemagglutinin (NTNH) protein having an amino acid sequence of SEQ ID NO: 3.

### CROSS-REFERENCE TO RELATED APPLICATION

The present application claims priority to and the benefit of U.S. Provisional Application Ser. No. 62/535,746 filed on Jul. 21, 2017, entitled “Compositions of Mosquitocidal Clostridial Proteins and Methods of Use,” the entire content of which is incorporated herein by reference.

### STATEMENT REGARDING FEDERALLY SPONSORED RESEARCH OR DEVELOPMENT

This invention was made with government support under Grant No. R01A1123390 and Grant No. 1R21A1070873 awarded by the National Institutes of Health. The government has certain rights in this invention.

### INCORPORATION BY REFERENCE

The instant application contains a Sequence Listing which has been submitted electronically in ASCII format and is hereby incorporated by reference in its entirety. Said ASCII copy, created on Jul. 20, 2018, is named 159654SEQLISTING.txt and is 81,949 bytes in size.

### BACKGROUND

Vector borne diseases and especially those transmitted by mosquitoes remain serious public health problems with constant threats of re-emergence. Mosquito-borne diseases have significantly impacted human civilization despite centuries of intensive control effort. Diseases such as dengue and Zika, filariasis and West Nile fever, and malaria are transmitted by infected mosquitoes of the genus Aedes, Culex, and Anopheles, respectively. All of these diseases remain serious public health problems.

Biological insecticides based on entomopathogenic bacteria such as Lysinibacillus sphaericus (Ls) and Bacillus thuringiensis israelensis (Bti) have been successfully used for decades as environmentally safe alternatives to control Culex and Aedes mosquito populations. Unfortunately, mosquito resistance to Ls has been noted in several areas due to overuse. Unlike Ls, no field resistance to Bti has yet been observed. Nonetheless, while the lack of resistance to Bti is fortunate, Bti does not effectively target Anopheles mosquitoes carrying malaria.

### SUMMARY

Aspects of embodiments of the present disclosure are directed to mosquitocidal compositions and methods of using the mosquitocidal compositions for eradicating (e.g., killing) or decreasing a population of Anopheles mosquitoes. The mosquitocidal compositions are derived from the toxin proteins of Clostridium bifermentans malaysia (Cbm) and Clostridium bifermentans Paraiba (Cbp).

In some embodiments of the present disclosure, a composition includes a microbe genetically modified to express a heterologous clostridial mosquitocidal protein 1 (CMP1) protein having an amino acid sequence of SEQ ID NO: 1 or a variant thereof and a heterologous non-toxic non-hemagglutinin (NTNH) protein having an amino acid sequence of SEQ ID NO: 3. In some embodiments, the microbe is not Clostridium bifermentans malaysia or Clostridium bifermentans paraiba. In some embodiments, the microbe is a bacterium, virus, yeast, or fungi. In some embodiments, the microbe may be the bacterium Lysinibacillus or Bacillus. For example, the bacterium may be Lysinibacillus sphaericus or Bacillus thuringiensis.

Additionally, in some embodiments of the present disclosure a composition includes a microbe genetically modified to express a heterologous clostridial mosquitocidal protein 1 (CMP1) protein having an amino acid sequence of SEQ ID NO: 1 or a variant thereof, a heterologous non-toxic non-hemagglutinin (NTNH) protein having an amino acid sequence of SEQ ID NO: 3, a heterologous OrfX1 protein having an amino acid sequence of SEQ ID NO: 5, a heterologous OrfX2 protein having an amino acid sequence of SEQ ID NO: 7, and/or a heterologous OrfX3 protein having an amino acid sequence of SEQ ID NO: 9. In some embodiments the microbe is genetically modified with a nucleic acid vector having an operon encoding ntnh-orfX1-orfX2-orfX3-cmp1. In some embodiments, the operon encoding ntnh-orfX1-orfX2-orfX3-cmp1 has a nucleic acid sequence of SEQ ID NO: 11.

According to some embodiments of the present disclosure, a mosquitocidal composition includes a CMP1 variant that is a homolog of the CMP1 protein having at least 85% identity with SEQ ID NO: 1 and capable of aligning with amino acid residues S1095, W1096, Y1097, and G1098 of SEQ ID NO: 1.

Some embodiments of the present disclosure are directed to a nucleic acid expression vector having a nucleic acid sequence encoding for a clostridial mosquitocidal protein 1 (CMP1) protein having an amino acid sequence of SEQ ID NO: 1 and a nucleic acid sequence encoding for a non-toxic non-hemagglutinin (NTNH) protein having an amino acid sequence of SEQ ID NO: 3. In some embodiments, the nucleic acid expression vector is capable of being transformed into a bacterium, virus, yeast, or fungus. In some embodiments, the nucleic acid expression vector also encodes for an OrfX1 protein having an amino acid sequence of SEQ ID NO: 5, an OrfX2 protein having an amino acid sequence of SEQ ID NO: 7, and/or an OrfX3 protein having an amino acid sequence of SEQ ID NO: 9.

Additionally in some embodiments of the present disclosure, a nucleic acid expression vector includes an operon encoding for NTNH having an amino acid sequence of SEQ ID NO: 3, ORFX1 having an amino acid sequence of SEQ ID NO: 5, ORFX2 having an amino acid sequence of SEQ ID NO: 7, ORFX3 having an amino acid sequence of SEQ ID NO: 9, and CMP1 having an amino acid sequence of SEQ ID NO: 1.

According to some embodiments of the present disclosure, a method of eradicating, (e.g., killing) or decreasing a population of an Anopheles mosquito species includes exposing or feeding a mosquitocidal composition according to embodiments of the present disclosure to Anopheles mosquito species. Non-limiting examples of Anopheles mosquito species include Anopheles gambiae, Anopheles coluzzi, Anopheles funestus, Anopheles darlingi, or Anopheles stephensi. For example, exposing may include spraying the presently disclosed mosquitocidal composition to an environment containing Anopheles mosquitoes.

Additionally, in some embodiments of the present disclosure, a method of killing an Anopheles mosquito species includes injecting a composition having a CMP1 protein having an amino acid sequence of SEQ ID NO: 1 or a variant thereof to the Anopheles mosquito species.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The accompanying drawings, together with the specification, illustrate example embodiments of the present disclosure, and, together with the description, serve to explain the principles of the present disclosure.

FIG. 1A is a plasmid map of the 109 kb megaplasmid in Clostridium bifermentans subsp. malaysia (“Cbm” or “Cb malaysia”). The outer scale is marked in base number from the predicted origin, the inner circle represents guanine-cytosine (GC) bias, with positive values in beige and negative values in purple; the 2nd circle from the center represents guanine and cytosine (G+C) content; the 3rd circle from the center are the toxin containing operons (pink), the cry and cmp operons are arrowed (the cry operon includes genes that encode proteinaceous insecticidal δ-endotoxins that form crystals and the cmp operon includes genes that encode a clostridial mosquitocidal protein); the 4th circle from the center are predicted genes on the forward strand (light blue); the 5th circle are predicted genes on the reverse strand (dark blue); the outer circle shows all genes encoded by the plasmid in both strands; color-coding for the genes is as follows: gray=regulatory; pink=toxin; blue=conserved hypothetical; red=unknown; green=transposon related; surface associated; Black=cell wall associated; yellow=miscellaneous metabolic genes, according to embodiments of the present disclosure.

FIG. 1B is a schematic depicting the configuration of clostridial neurotoxin loci in different bacterial strains as indicated and in Cbm and Clostridium bifermentans paraiba (“Cbp” or “Cb Paraiba”). The ctox locus, which encodes the clostridial mosquitocidal protein 1 (CMP1) protein, in Cbm and Cbp consists of the CMP operon and two genes, p47 and ha41, with IS and flagella (fla′) sequences flank these loci; Bont/=botulinum neurotoxin; ntnh=non-toxic non-hemagglutinin; ha=hemagglutinin; orfX, p47=proteins of unknown function, according to embodiments of the present disclosure.

FIG. 2A is a graph showing the toxicity (% mortality) of CMP1 in Aedes aegypti (black squares), CMP1 in Anopheles coluzzi (red circles) and catalytically inactive CMP1 E209Q mutant in Aedes aegypti (blue triangles) mosquito larvae by injection dose (amol/larva), where the data points represent the average of the percentage of mortality of at least two biological replicates of 15 larvae, according to embodiments of the present disclosure.

FIG. 2B is a schematic depicting nucleic acid constructs expressing the proteins included in CMP operon (left panel) and their corresponding mortality to 3rd instar A. aegypti and An. coluzzi larvae after 3 days of exposure; with all constructs having a Cry3A promoter from B. thuringiensis tenebrionis (Cry3A P) or a Cyt1A promoter from B. thuringiensis israelensis (Cyt1A P) and Cry1A stem loop terminator (Cry1A SL); where for expression of cmp1 gene in NTNH-CMP1 and orfX1, orfX2, orfX3, cmp1 genes in NTNH-OrfX1-3-CMP1 construct the native Shine-Dalgarno sequences was used; and error bars represent ±S.D. of three different experiments, according to embodiments of the present disclosure.

FIG. 3 is a table of toxicity of Cb malaysia, Cb paraiba, and B. thuringiensis israelensis (Bti) in 3rd instar Aedes aegypti, Anopheles coluzzi and Anopheles stephensi mosquito larvae and a mixture of different instars of Drosophila melanogaster larvae; where LC₅₀ (the lethal concentration required to kill 50% of a population) is represented as volume of whole culture in 100 ml water and in CFU/ml, according to embodiments of the present disclosure.

FIG. 4 is a table of sequencing data of the Cb malaysia predicted genes, according to embodiments of the present disclosure.

FIG. 5 is a graph depicting the gene functional annotation of Cb malaysia genome; where annotated genes were aligned with Clusters of Orthologous Groups (COG) function classification database, as indicated from B to V where: B is Chromatin structure and dynamics; C is Energy production and conversion; D is Cell cycle control, cell division, chromosome partitioning; E is Amino acid transport and metabolism; F is Nucleotide transport and metabolism; G is Carbohydrate transport and metabolism; H is Coenzyme transport and metabolism; I is Lipid transport and metabolism; J is Translation, ribosomal structure and biogenesis; K is Transcription; L is Replication, recombination and repair; M is Cell wall/membrane/envelope biogenesis; N is Cell motility; 0 is Posttranslational modification, protein turnover, chaperones; P is Inorganic ion transport and metabolism; Q is Secondary metabolites biosynthesis, transport and catabolism; R is General function prediction only; S is an Unknown function; T is Signal transduction mechanism; U is Intracellular trafficking, secretion and vesicular transport; and V is defense mechanisms, according to embodiments of the present disclosure.

FIG. 6 is a table showing the presence of plasmids (marked with X) in the indicated Clostridium bifermentans (Cb) mosquitocidal and non mosquitocidal strains, according to embodiments of the present disclosure.

FIG. 7A is a schematic of a neighbor joining phylogenetic tree generated from the gene codon sequences of different clostridial neurotoxins and Cbm CMP1 and NTNH using MEGA software, according to embodiments of the present disclosure.

FIG. 7B shows alignment of the C-terminus of CMP1 and the indicated Botulinum neurotoxins, showing the conserved SxWY ganglioside binding site, according to embodiments of the present disclosure.

FIG. 7C shows alignment of a LC fragment from CMP1 and different clostridial neurotoxins, showing the conserved motif HELXH in the catalytic site, according to embodiments of the present disclosure.

FIG. 8A is a Western blot in an SDS-PAGE gel of the CMP1 protein immunodetected using a CMP1 heavy chain antibody in the Cbm culture, but not in the Cbm loss-of-function mutant CbmA109, or in the type strain Cb, according to embodiments of the present disclosure.

FIG. 8B is a fractionation scheme to isolate the toxin complex, in which the fraction obtained by citrate extraction retains toxicity to Anopheles, according to embodiments of the present disclosure.

FIG. 8C is a Western blot of CMP1 protein and Cry16 protein in an SDS-PAGE gel of the Cb malaysia extracted fraction, according to embodiments of the present disclosure.

FIG. 8D is native PAGE gel of Cbm, Cb, and CbmΔ109 extracted fractions, where the lanes were split in two samples (E1 and E2) for mass spectrometry analysis, according to embodiments of the present disclosure.

FIG. 8E is a Western blot of a Native PAGE gel of a Cbm extracted fraction (left lane) and whole culture of B. thuringiensis expressing a NTNH-OrfX1-3-CMP1 construct (right lane), showing similar sizes are observed in both the fraction and the whole culture, according to embodiments of the present disclosure.

FIG. 9 is a table of proteins identified by mass spectrometry from the Cb malaysia extracted fraction encoded by the 109, 7.2 and 4 kb Cb malaysia and Cb paraiba plasmids, organized by score, with the proteins from cry and Ctox toxin loci highlighted in yellow, according to embodiments of the present disclosure.

FIG. 10A is a graph showing the motion of 15 3rd instar A. aegypti larvae individuals (points) after water (control), CMP1, or inactive CMP1 E209Q mutant injection as indicated with the number of larval lashing movements shown for a 30 second period, where the boxes represent the middle 50% of the data, the line in the middle of the box represents the median, the box edges are the 25th and 75th percentiles and the vertical lines the min and max values, according to embodiments of the present disclosure

FIG. 10B shows graphs of the percentage of the indicated adult mosquitoes and flies that stopped flying after 24 hours of injection of CMP1, with the injury rate produced by the injection itself (dead individuals 1 h after injection) indicated above each group and being independent of the dose injected, with the following number of injections: 58 Aedes control, 60 4 pg CMP1, 43 100 pg CMP1; 54 Anopheles control, 65 4 pg CMP1, 62 100 pg CMP1; and 15 Drosophila control, 15 100 pg CMP1, according to embodiments of the present disclosure.

FIG. 10C is a graph showing the decrease of CMP1 toxicity produced by the pre-incubation of 0.4 ng/ul of toxin with 5 mM 1,10-phenanthroline before injection, where a decrease is represented as percentage in comparison to the injection of CMP1 without inhibitor, and error bars represent ±S.D. of three replicates of 15 individuals, according to embodiments of the present disclosure.

FIG. 11A is a representation of the recombinant soluble NSF (N-ethylmaleimide-sensitive factor) attachment protein receptors (SNARE proteins) fused to GST or a His-tag in the N terminus and a myc tag in C terminus used in CMP1 LC cleavage assays (upper panel); with immunodetection of SNARE proteins and syntaxin mutants in the absence or in presence of CMP1 LC or CMP1 catalytically inactive E209Q mutant using GST, syntaxin, His and myc antibodies (lower panel), according to embodiments of the present disclosure.

FIG. 11B is an SDS-PAGE of His-labeled syntaxin cleavage assay showing the fragment of 4.5 KDa band released from the cleavage by CMP1 LC, according to embodiments of the present disclosure.

FIG. 11C is a mass spectrum of the HAMDYVQTATQDTKK (SEQ ID NO: 39) peptide from His-syntaxin found in the sample, according to embodiments of the present disclosure.

FIG. 11D shows the His-syntaxin amino acid sequence (highlighted in blue) which was detected by mass spectrometry upon incubation with CMP1 LC which was not found in the control sample or the sample incubated with CMP1 E209Q mutant, according to embodiments of the present disclosure.

FIGS. 11E-11G are each a mass spectrum of the HAMDYVQTATQDTKK (SEQ ID NO: 39) peptide, the ALKYQSEQKLISE (SEQ ID NO: 40) peptide, or the LEQKLISEEDL (SEQ ID NO: 41) peptide as indicated from His-syntaxin, according to embodiments of the present disclosure.

FIG. 11H is the amino acid sequence of the C-terminus of An. gambiae syntaxin or human syntaxin, as indicated, where the amino acids that are not conserved in mosquitoes in comparison to human syntaxin are highlighted in red, and the position of the cleavage site by CMP1 LC and the mutations introduced in An. gambiae syntaxin and tested in cleavage assays are indicated with arrows, according to embodiments of the present disclosure

### DETAILED DESCRIPTION

The anaerobic bacterium Clostridium bifermentans subsp. malaysia (referred to herein as “Cbm” or “Cb malaysia”) shows high mosquitocidal activity, primarily to Anopheles mosquito larvae—the vector of malaria, while the Cb type strain is not mosquitocidal. Additionally, Cbm is innocuous to mammals, fish, and non-target invertebrates rendering suitable applications safe to use on disease-carrying Anopheles mosquitoes in the proximity of people. Nonetheless, until now, the lack of knowledge about the mechanism of toxicity has precluded the use of this bacterium as a bioinsecticide.

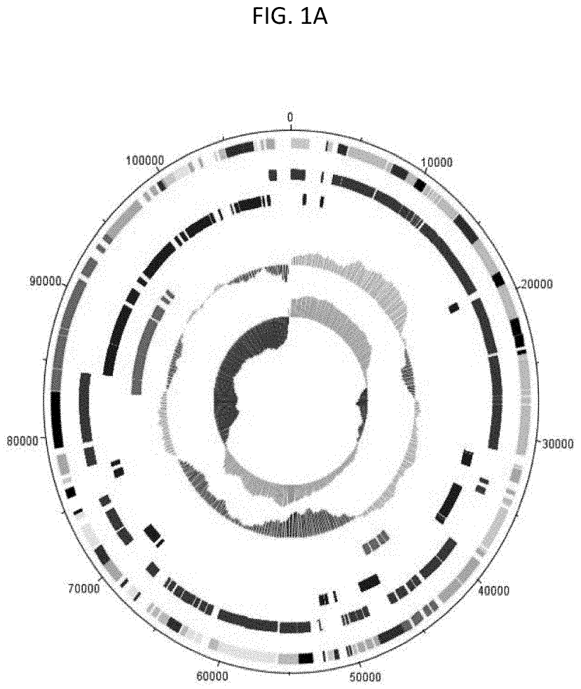

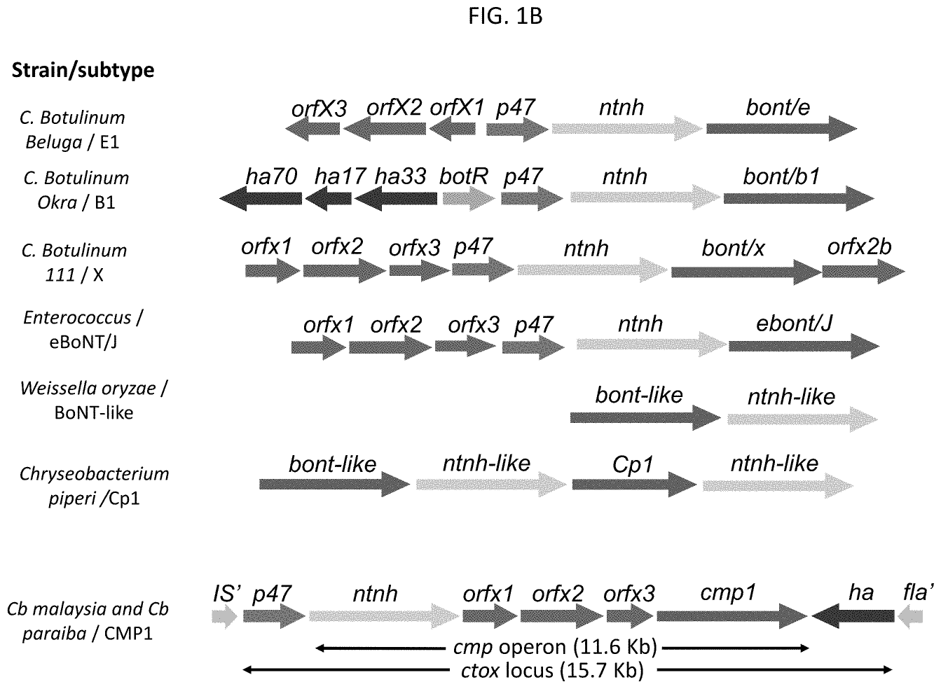

With reference to FIG. 1A, comparative genomics of two Clostridium bifermentans (Cb) mosquitocidal strains Cb malaysia (Cbm) as well as Cb paraiba (Cbp) with the non-mosquitocidal Cb type strain, identified a megaplasmid of 109 kilobases (kb) found in both the Cbm and Cbp mosquitocidal strains that was not found in the non-mosquitocidal Cb type strain. A map of the 109 kb plasmid is depicted in FIG. 1A. Analysis of the 109 kb plasmid resulted in the identification of a toxin gene locus referred to as ctox.

With reference to FIG. 1B, the ctox locus of 15.7 kb encodes a protein referred to as the clostridial mosquitocidal protein 1 (CMP1) protein for its similarity to clostridial neurotoxins (CNTs) (e.g., BoNT proteins). Additionally, the cmp1 gene is found in an operon (e.g., under the control of the same promoter) with orfx1, orfx2, orfx3, and non-toxic non-hemagglutinin (ntnh) genes (FIG. 1B).

Based on the mosquitocidal analysis of the proteins expressed in the cmp1 operon, aspects of embodiments of the present disclosure include a composition having a heterologously expressed CMP1 protein or a variant thereof. Some compositions of the present disclosure may include a heterologously expressed CMP1 protein or a variant thereof and a heterologously expressed NTNH protein. Some compositions of the present disclosure may include a heterologously expressed CMP1 protein or a variant thereof, a heterologously expressed NTNH protein, and heterologously expressed OrfX1, OrfX2, and OrfX3 proteins.

For effective introduction and distribution of a mosquitocidal composition into a population of Anopheles mosquitoes, a genetically modified host microbe may be used. Accordingly, in some embodiments, a composition includes a microbe transformed to express a CMP1 protein or a variant thereof, a CMP1 protein or a variant thereof and an NTNH protein, or a CMP1 protein or a variant thereof, an NTNH protein, and the OrfX1, OrfX2, and OrfX3 proteins. Suitable microbes include any bacterium, virus, yeast, or fungus that has been characterized in the art for genetic modification. For example, a suitable microbe has established methods for transformation of and protein expression from a nucleic vector encoding one or more of the heterologous proteins from the CMP1 operon. In some embodiments, the host microbe is any non-mosquitodical Clostridium bifermentans strain, and therefore is not Clostridium bifermentans malaysia (Cbm) or Clostridium bifermentans paraiba (Cbp). Additionally, suitable microbes also include the bacterium Lysinibacillus or Bacillus. For example, Lysinibacillus sphaericus or Bacillus thuringiensis.

As used herein, “CMP1” refers to the Cbm CMP1 protein having an amino acid sequence of SEQ ID NO: 1. Accordingly, for heterologous expression of a CMP1 protein of SEQ ID NO: 1, the corresponding DNA sequence encoding for the CMP1 protein may be synthesized for codon bias and subcloned into any suitable nucleic acid expression vector for transformation and expression in a suitable host microbe. For example, for heterologous expression of the CMP1 protein in Bacillus thuringiensis, the cmp1 DNA construct of SEQ ID NO: 2 may be used in a nucleic acid expression vector suitable for transformation and expression in Bacillus thuringiensis.

As used herein, “NTNH” refers to Cbm NTNH protein having an amino acid sequence of SEQ ID NO: 3. Accordingly, for heterologous expression of a NTNH protein of SEQ ID NO: 3, the corresponding DNA sequence encoding for the NTNH protein may be synthesized for codon bias and subcloned into any suitable nucleic acid expression vector for transformation and expression in a suitable host microbe. For example, for heterologous expression of the NTNH protein in Bacillus thuringiensis, the ntnh DNA construct of SEQ ID NO: 4 may be used in a nucleic acid expression vector suitable for transformation and expression in Bacillus thuringiensis.

As used herein, each of “OrfX1,” “OrfX2,” and “OrfX3” refers to Cbm OrfX1 protein, Cbm OrfX2 protein, and Cbm OrfX3 protein, respectively. OrfX1 has an amino acid sequence of SEQ ID NO: 5. Accordingly, for heterologous expression of the OrfX1 protein of SEQ ID NO: 5, the corresponding DNA sequence encoding for the OrfX1 protein may be synthesized for codon bias and subcloned into any suitable nucleic acid expression vector for transformation and expression in a suitable host microbe. For example, for heterologous expression of the OrfX1 protein in Bacillus thuringiensis, the orfX2 DNA construct of SEQ ID NO: 6 may be used in a nucleic acid expression vector suitable for transformation and expression in Bacillus thuringiensis. OrfX2 has an amino acid sequence of SEQ ID NO: 7. Accordingly, for heterologous expression of the OrfX2 protein of SEQ ID NO: 7, the corresponding DNA sequence encoding for the OrfX2 protein may be synthesized for codon bias and subcloned into any suitable nucleic acid expression vector for transformation and expression in a suitable host microbe. For example, for heterologous expression of the OrfX2 protein in Bacillus thuringiensis, the orfX2 DNA construct of SEQ ID NO: 8 may be used in a nucleic acid expression vector suitable for transformation and expression in Bacillus thuringiensis. OrfX3 has an amino acid sequence of SEQ ID NO: 9. Accordingly, for heterologous expression of the OrfX3 protein of SEQ ID NO: 9, the corresponding DNA sequence encoding for the OrfX3 protein may be synthesized for codon bias and subcloned into any suitable nucleic acid expression vector for transformation and expression in a suitable host microbe. For example, for heterologous expression of the OrfX3 protein in Bacillus thuringiensis, the orfX3 DNA construct of SEQ ID NO: 10 may be used in a nucleic acid expression vector suitable for transformation and expression in Bacillus thuringiensis.

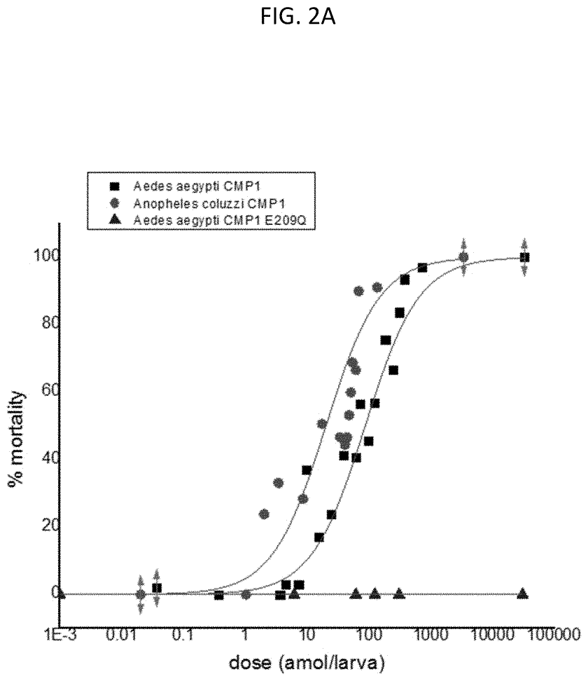

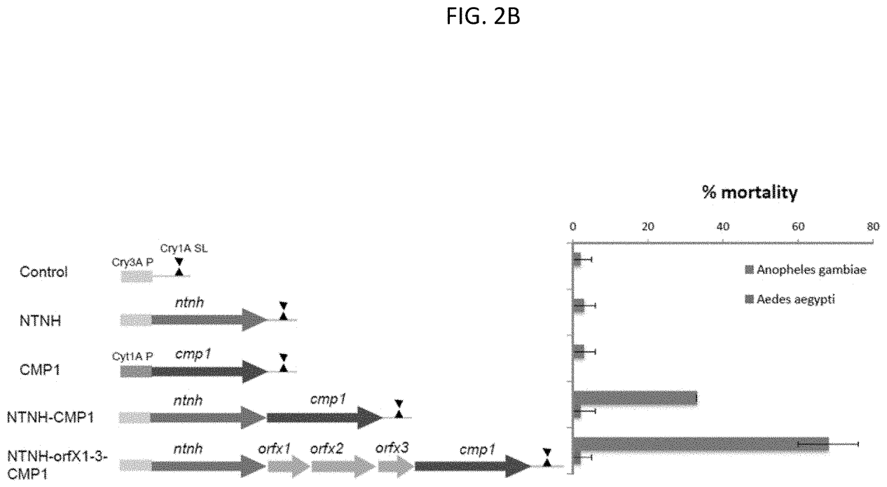

With reference to FIG. 2A, purified CMP1 protein shows high toxicity when injected directly into mosquito larvae. However, as shown in FIG. 2B, mosquito larvae exposed to a host microbe (e.g., B. thuringiensis) expressing CMP1 does not show toxicity. Without being bound by any theory, CMP1 ingested through exposure of a host microbe may not be capable of being absorbed by the mosquito and is therefore not toxic. However, with reference to FIG. 2B, CMP1 expressed together with NTNH in a host microbe results in mosquitocidal activity, and CMP1 expressed together with NTNH and OrfX1, OrfX2, and OrfX3 in a host microbe results in higher mosquitocidal activity. Accordingly, in some embodiments, a composition of the present disclosure includes a microbe genetically modified to express a heterologous CMP1 protein and a heterologous NTNH protein. In some embodiments, a composition of the present disclosure includes a microbe genetically modified to express a heterologous CMP1 protein, a heterologous NTNH protein, a heterologous OrfX1 protein, a heterologous OrfX2 protein, and a heterologous OrfX3 protein.

In some embodiments of the present disclosure, a composition includes a microbe genetically modified with the cmp1 operon of ntnh, orfX1, orfX2, orfX3, and cmp1. The cmp1 operon has a DNA sequence of SEQ ID NO: 11. Accordingly, for heterologous expression of NTNH, OrfX1, OrfX2, OrfX3, and CMP1, the corresponding DNA sequence of SEQ ID NO: 11 encoding for these proteins may be subcloned into any suitable nucleic acid expression vector for transformation and expression in a suitable host microbe. For example, for heterologous expression of the proteins of the cmp1 operon in Bacillus thuringiensis, the cmp1 operon DNA construct of SEQ ID NO: 11 may be used in a nucleic acid expression vector suitable for transformation and expression in Bacillus thuringiensis. In some embodiments, the cmp1 operon has DNA sequence that is codon optimized from SEQ ID NO: 11. Accordingly, for heterologous expression of the proteins of the cmp1 operon a DNA sequence encoding for NTNH(SEQ ID NO: 3)-OrfX1 (SEQ ID NO:5)-OrfX2 (SEQ ID NO: 7)-OrfX3 (SEQ ID NO:9)-CMP1 (SEQ ID NO:1) may be subcloned into a suitable nucleic acid expression vector for transformation and expression in a suitable host microbe.

Abbreviations for amino acids are used throughout this disclosure and follow the standard nomenclature known in the art. For example, as would be understood by those of ordinary skill in the art, Alanine is Ala or A; Arginine is Arg or R; Asparagine is Asn or N; Aspartic Acid is Asp or D; Cysteine is Cys or C; Glutamic acid is Glu or E; Glutamine is Gln or Q; Glycine is Gly or G; Histidine is His or H; Isoleucine is Ile or I; Leucine is Leu or L; Lysine is Lys or K; Methionine is Met or M; Phenylalanine is Phe or F; Proline is Pro or P; Serine is Ser or S; Threonine is Thr or T; Tryptophan is Trp or W; Tyrosine is Tyr or Y; and Valine is Val or V.

As used herein “variant thereof” as in “CMP1 or a variant thereof” refers to a homolog or fragment of the referenced gene (e.g., CMP1 (SEQ ID NO: 1) having at least 50% of the mosquitodical activity of CMP1). For example, a homolog or fragment of CMP1 has at least 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% of the mosquitodical activity of CMP1. A homolog of CMP1 having at least 50% up to 99% of the mosquitocidal activity of CMP1 refers to a protein homolog sharing an overall amino acid sequence identity of at least 85% with CMP1 (SEQ ID NO: 1) and the protein homolog shares alignment with amino acid residues S1095, W1096, Y1097, and G1098 of SEQ ID NO: 1. For example, the amino acid residues S1095, W1096, Y1097, and G1098 of SEQ ID NO:1 may not occur at the same residue number in the amino acid sequence of the protein homolog, but all of these consecutive amino acids of S1095, W1096, Y1097, and G1098 are present in the protein homolog sharing at least 85% overall amino acid identity with the CMP1 (SEQ ID NO:1) and are capable of being aligned with S1095, W1096, Y1097, and G1098 of SEQ ID NO: 1.

In some embodiments, homologs of CMP1 having at least 85% homology to SEQ ID NO:1 and having alignment with amino acid residues S1095, W1096, Y1097, and G1098 of SEQ ID NO: 1 may include conservative amino acid substitutions of SEQ ID NO:1. For example, conservative amino acid substitutions include: substitution of Y with F; T with S, K, or A; P with A; E with D or Q; N with D or G; R with K; G with N or A; T with S, K, or A; D with N or E, I with L or V, F with Y or L; S with T or A, R with K, G with N or A, K with R; A with S, K, P, G, T, or V; W with Y; and M with L.

In some embodiments of the present disclosure, a method of killing or decreasing a population of an Anopheles mosquito species includes injecting the Anopheles mosquito species with a composition of the present disclosure having a heterologously expressed CMP1 protein or a variant thereof.

In some embodiments of the present disclosure, a method of killing or decreasing a population of an Anopheles mosquito species includes exposing (e.g., incubating or spraying) the Anopheles mosquito species with a composition of the present disclosure including a microbe genetically modified (e.g., by transformation with a nucleic acid expression vector) to express heterologous CMP1 protein (SEQ ID NO: 1) or a variant thereof and a heterologous NTNH protein (SEQ ID NO: 3). Exposing the Anopheles mosquito species to a composition of the present disclosure by spraying includes feeding the composition to the mosquito as spraying may result in providing a composition of the present disclosure to the surface of a food source for the Anopheles mosquito species. In some embodiments, a method of killing or decreasing a population of an Anopheles mosquito species includes exposing the Anopheles mosquito species with a composition of the present disclosure including a microbe genetically modified to express heterologous CMP1 protein (SEQ ID NO: 1) or a variant thereof, a heterologous NTNH protein (SEQ ID NO: 3), a heterologous OrfX1 protein (SEQ ID NO: 5), a heterologous OrfX2 protein (SEQ ID NO: 7), and a heterologous OrfX3 protein (SEQ ID NO: 9). Spraying of the composition, for example, may include spraying the composition or administering the composition to an environment containing Anopheles mosquitoes.

According to embodiments of the present disclosure, methods for killing or decreasing a population of Anopheles mosquitoes include any species of Anopheles mosquitoes. Non-limiting examples of Anopheles mosquitoes include Anopheles gambiae, Anopheles coluzzi, Anopheles funestus, Anopheles darlingi, or Anopheles stephensi.

The following examples are presented for illustrative purposes only, and do not limit the scope or content of the present application.

### EXAMPLES

### Example 1. Genome Sequencing of C. bifermentans Strains

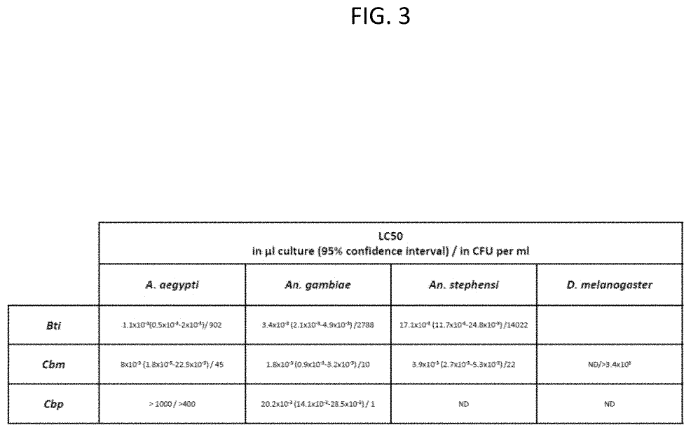

To identify new Cb mosquitocidal components, the genomes of two Cb mosquitocidal strains Cbm and Cb paraiba (Cbp) were sequenced which show higher selectivity to Anopheles than Aedes mosquitoes (FIG. 3) and the non-mosquitocidal Cb.

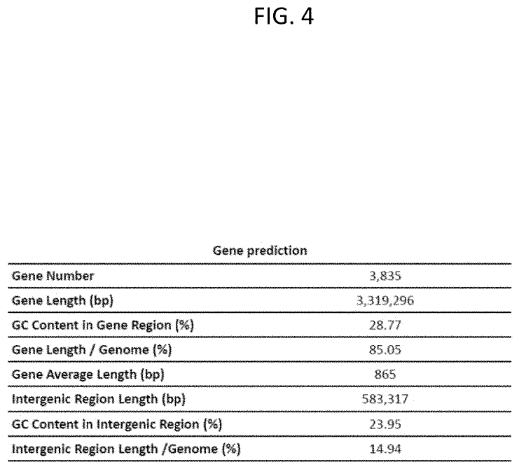

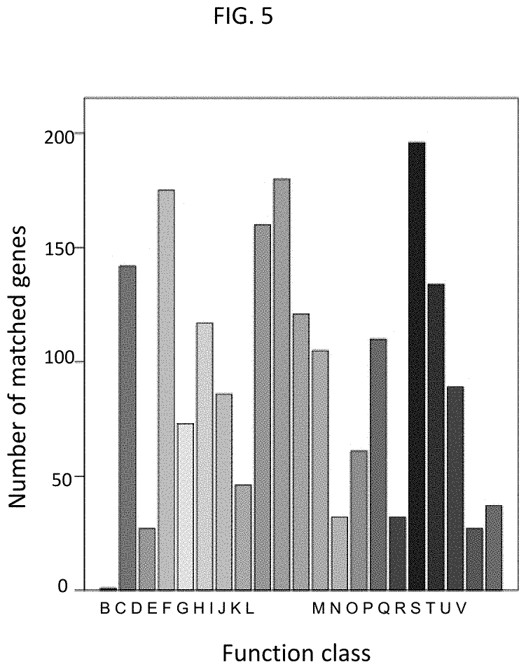

The Cbm chromosome is approximately 3.6 Mbp and encodes 3465 predicted protein-coding genes (FIG. 4 and FIG. 5) Cbm, Cbp, and Cb genomes have a similar chromosome sizes and belong to the group of extremely low GC (guanine and cytosine) content in clostridia, with 28% GC content (FIG. 4).

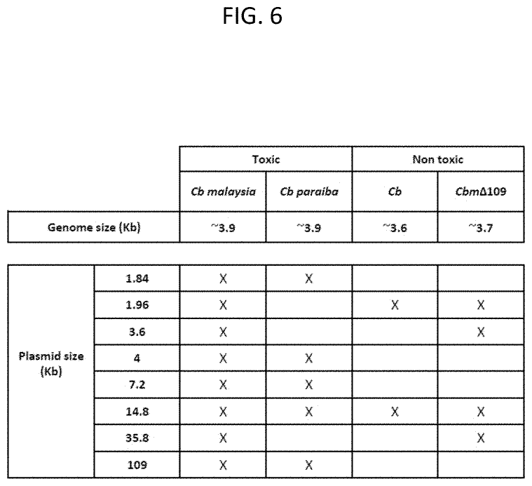

Eight extra scaffolds from Cbm sequencing data did not match chromosomic sequences. PCR amplification from the scaffolds' ends confirmed their circularity and these scaffolds represent the eight plasmids in this strain, although an earlier report indicated that this strain did not contain plasmids (Seleena P, et al., 1997, J Am Mosq Control Assoc. 3(4): 395-7, the entire content of which is incorporated herein by reference), but another reported that it contain 5 plasmids smaller than 20 kb (Barloy et al., 1998, Gene 211: 293-299, the entire content of which is incorporated herein by reference). Similarly, the five Cbp and two Cb plasmids were confirmed by PCR. Notably the mosquitocidal strains shared 4 plasmids, which were not present in non mosquitocidal Cb (FIG. 6).

### Example 2. The Toxicity of Cbm is Linked to a Plasmid with Two Toxin Loci

To obtain a loss of function mutant, Cbm cells were irradiated with cesium-137. Out of more than 3000 colonies screened, three completely (or substantially completely) lost their activity (or their observable activity) against Aedes aegypti and Anopheles stephensi mosquito larvae. One mutant, CbmΔ109, was selected and genome sequenced. Comparison of Cbm and CbmΔ109 genomes showed that the non-toxic mutant had lost 4 Cbm plasmids which are also present in Cbp (FIG. 6).

These four plasmids in Cbm and Cbp that are absent in non-mosquitocidal Cb and the CbmΔ109 mutant likely code toxin genes. Since three less than (<) 8 kb plasmids coded for genes that did not appear to be toxigenic, the largest plasmid of 109 kb was analyzed (FIG. 1A). The proteins encoded in the Cbm and Cbp 109 kb plasmids were annotated and summarized in the attached APPENDIX. The 109 kb plasmid contains several uncharacterized putative genes, transposons, and insertion sequences as well as genes encoding for cell wall-associated hydrolases, replication proteins, and a type IV secretion system. Additionally, cry16A, cry17A, and the two hemolysin-like genes were identified in a cry operon (e.g., genes that encode proteinaceous insecticidal δ-endotoxins that form crystals), which were previously implicated in Aedes as disclosed in Barloy et al., 1998, supra, but not in Anopheles toxicity as described in Qureshi et al., 2014, Appl Environ Microbiol 80 (18):5689-5697, the entire content of which is incorporated herein by reference.

The second toxin locus downstream of the cry operon (FIG. 1A) and flanked by insertion sequences and transposon elements was named ctox. The ctox locus encodes a protein with similarity to clostridial neurotoxins (CNTs), a group which includes the tetanus neurotoxin (TeNT) produced by C. tetani and botulinum neurotoxins (BoNT) produced by C. botulinum (groups I-IV) and some strains of C. butyricum and C. baratii. The gene which codifies for the CNT was named Clostridial Mosquitocidal Protein 1 (CMP1). Adjacent to the cmp1 gene were additional genes encoding for non-toxic non hemagglutinin (NTNH), hemagglutinin (HA), OrfX1, OrfX2, OrfX3 and P47 proteins (FIG. 1B).

With the exception of BoNT/C and D—which are more related to avian and cattle botulism—most of the characterized CNTs are reported to be toxic to humans. The toxicity of CNTs is primarily by ingestion, thus the toxins endure extreme pH and potential proteolysis in the gut (e.g., digestive system) to reach the bloodstream and from there the nerve terminal targets, where after receptor binding, the toxin light chain (LC) undergo endocytosis and cleaves target SNARE proteins. In the gut, the CNTs travel as high molecular weight complexes with associated protein components, like NTNH and HA, which have been reported to stabilize the toxin. Additionally, HA proteins have also been involved in epithelial barrier disruption. However, the function of OrfX proteins remains unknown.

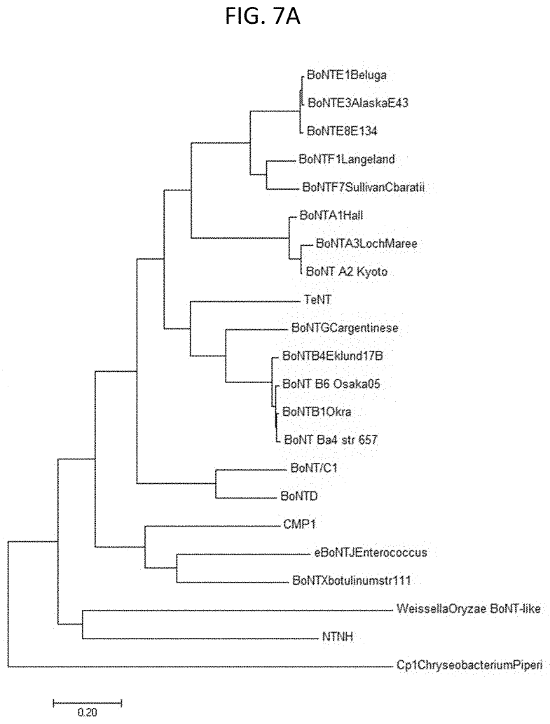

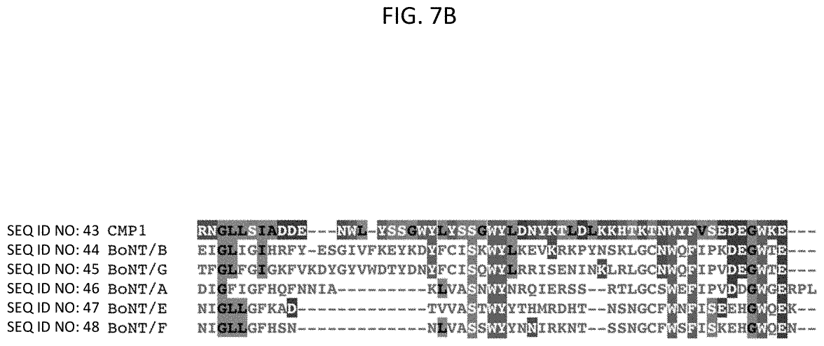

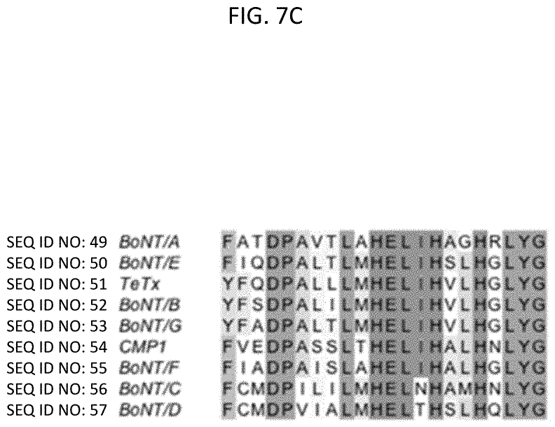

The Cbm NTNH, OrfX1-3, CMP1 and P47 proteins have 35 to 57% amino acid identity to Clostridium proteins. In contrast, Cbm HA is quite divergent from the Clostridium HAs but related to Paenibacillus sp. hemagglutinins (45% identity). The closest known relative to CMP1 is BoNT/X from C. botulinum strain 111 (36% identity), followed by Enterococcus BoNT-like protein (34% identity) (FIG. 7A). The SxWY motif in the binding domain (HC) of CMP1 is conserved, which in BoNTs is involved in ganglioside receptor binding (FIG. 7B), and the conserved cysteines are implicated in the disulfide bond that links the toxin heavy and LC. The zinc-dependent protease motif HExxH which confers the LC metalloprotease activity that cleaves target SNARE proteins in the neuron cytosol is also conserved (FIG. 7C).

The Ctox locus shows a novel gene organization with an OrfX1-3 gene cluster in the same orientation as NTNH and the CNT, as observed in Enterococcus BoNT-like and BoNT/X encoding strains, but in Cbm and Cbp it is located between NTNH and CMP1 under the same promoter (FIG. 1B). This configuration suggests that the horizontal gene transfer to Cbm or Cbp likely occurred from an ancestral bacterium as it has also been speculated for the Enterococcus BoNT-like cluster.

### Example 3. The Cmp Operon Proteins Show Oral Toxicity to Anopheles Mosquito Larvae

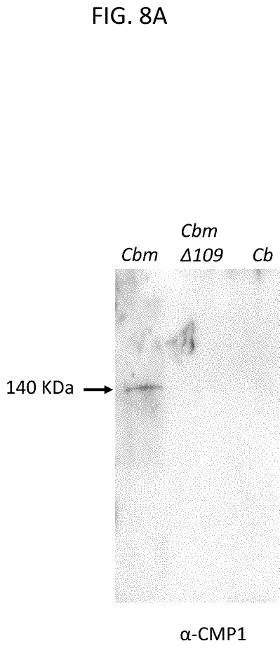

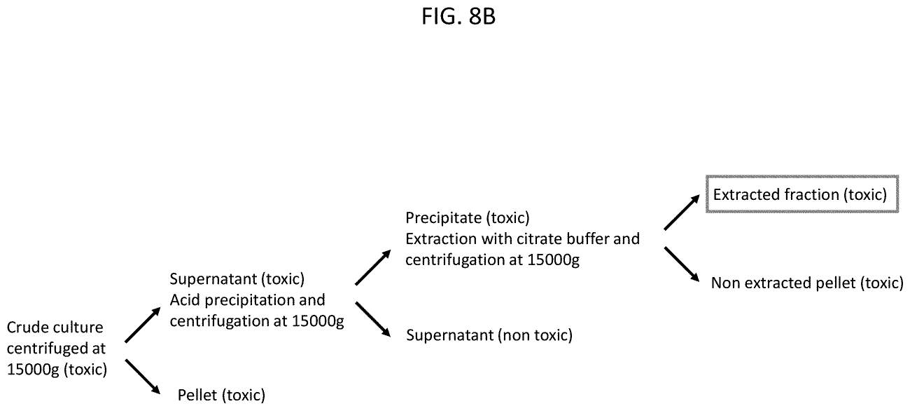

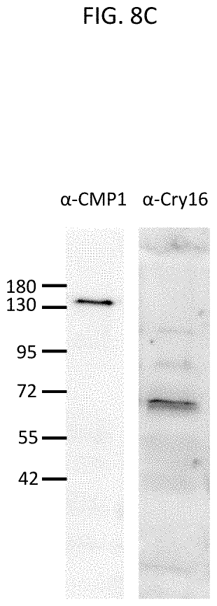

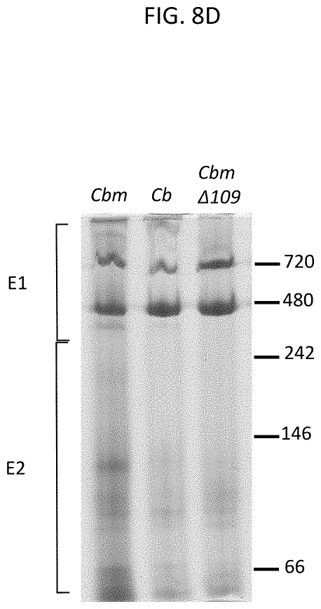

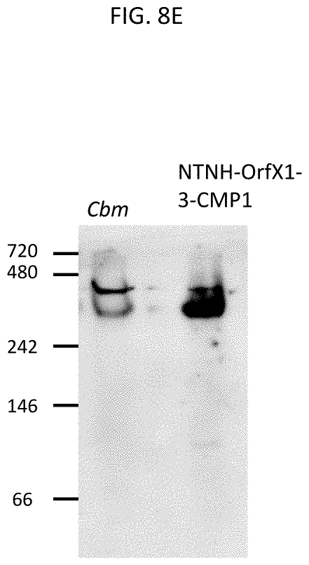

CMP1 was immunodetected as a 145 kDa protein in Cbm cultures (FIG. 8A). In order to concentrate high molecular complexes produced by Cbm, culture proteins were acid precipitated and extracted in sodium citrate buffer as outlined in FIG. 8B and as described for concentration of botulinum neurotoxin complexes in Lin et al., 2015, Appl Environ Microbiol. 81(2):481-91, the entire content of which is incorporated herein by reference. The extracted fraction, which retained toxicity to Anopheles and contained CMP1 and Cry16A (FIG. 8C), was separated by native acrylamide gels, subjected to analysis by UPLC/MS/MS and compared with a similar extracted fraction from CbmΔ109 mutant (FIG. 8D and FIG. 8E, first lane).

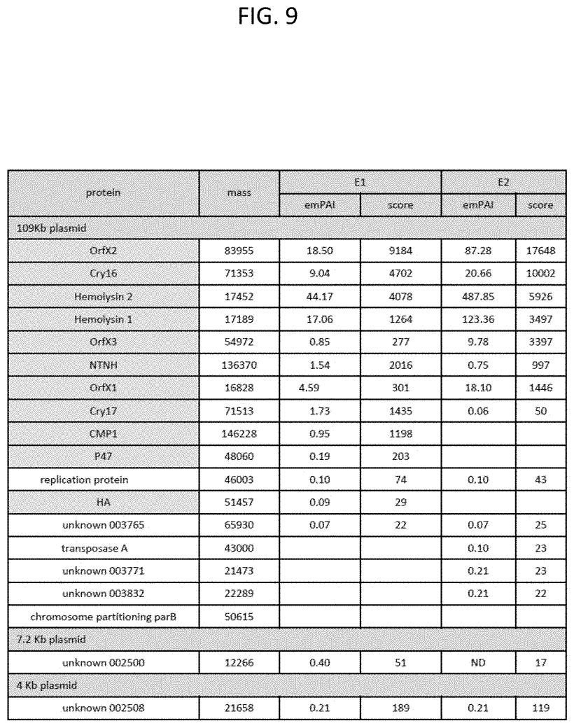

All proteins from the cry and ctox loci were detected in the Cbm sample (FIG. 9) but as expected, absent in CbmΔ109. Only a few proteins which are not expected to be toxigenic were found to be encoded by the 109 kb, 7.2 kb, and 4 kb Cbm plasmids (FIG. 9).

To verify that the cmp1 operon encodes the anopheline active toxins, the ntnh, orfX1, orfX2, orfX3 and cmp1 genes were cloned in pHT315 shuttle vector in different combinations and transformed into B. thuringiensis (Bt) 4Q7 strain (Bacillus Stock Center, Ohio State University, Columbus, Ohio). The constructs were tested for toxicity using An. coluzzi and Ae. aegypti larvae, as shown in FIG. 2B. After 3 days of exposure, the Bt cultures expressing either the CMP1 or NTNH protein alone had no toxicity to An. coluzzi. However, cultures expressing both NTNH and CMP1 proteins showed 33% mortality, whereas the one expressing the full operon (FIG. 8E) raised the mortality up to 70% (FIG. 8B). Accordingly, the OrfX1-3 and NTNH proteins in the operon enhance CMP1 toxicity. None of the constructs were significantly toxic to Ae. aegypti.

### Example 4. CMP1 is Toxic to Mosquito Larvae In Vivo

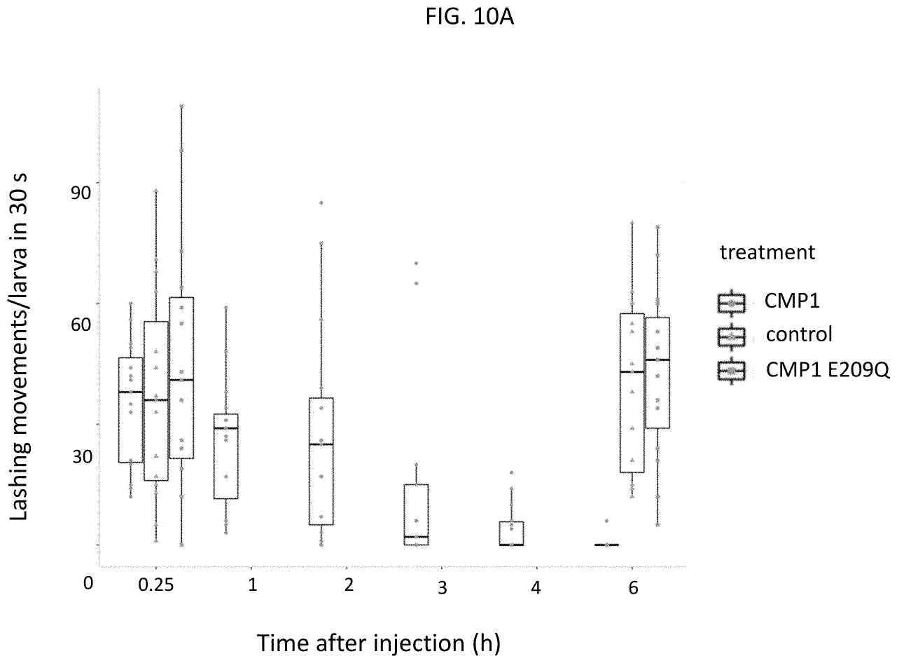

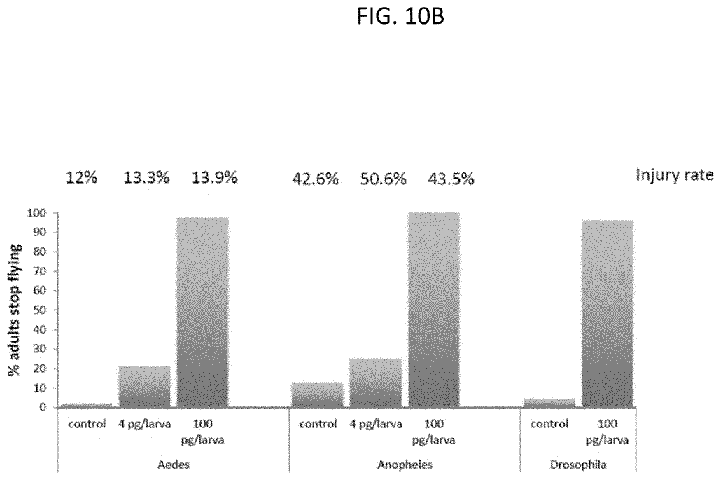

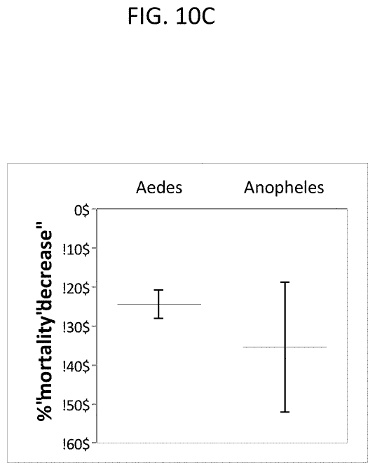

In order to evaluate if CMP1 alone is toxic when the gut barrier is bypassed, recombinant CMP1 was injected into mosquito larvae. With reference to FIG. 8A, injected CMP1 was highly toxic to both Aedes and Anopheles mosquito species after 24 hours, with an LD₅₀ (the amount of an administered substance that kills 50 percent of a population) of 14 pg (98 amol) and 6.5 pg (44.5) amol per larva, respectively. Additionally, Aedes larvae injected with the LC₁₀₀ (54 pg/larva) fully recovered 15 minutes after injection, but at 3 hours, the larvae showed significant slowing of motion (FIG. 10A) which is consistent with the paralysis associated with CNTs' intoxication. With reference to FIG. 10B, CMP1 was also toxic to adult mosquitoes of both species by injection, since after 24 hours a dose-dependent impairment in their ability to fly was observed. Pre-incubation of the toxin with the metalloprotease inhibitor 1,10-phenanthroline before larval injection decreased CMP1 toxicity as shown in FIG. 10C. Furthermore, with reference to FIG. 8A, the mutation E209Q in the putative metalloprotease active site (HExxH motif) abolished the mosquitodical activity completely indicating that CMP1 is a metalloprotease and this activity is significant or essential for toxicity.

### Example 5. CMP1 Cleaves Mosquito Syntaxin

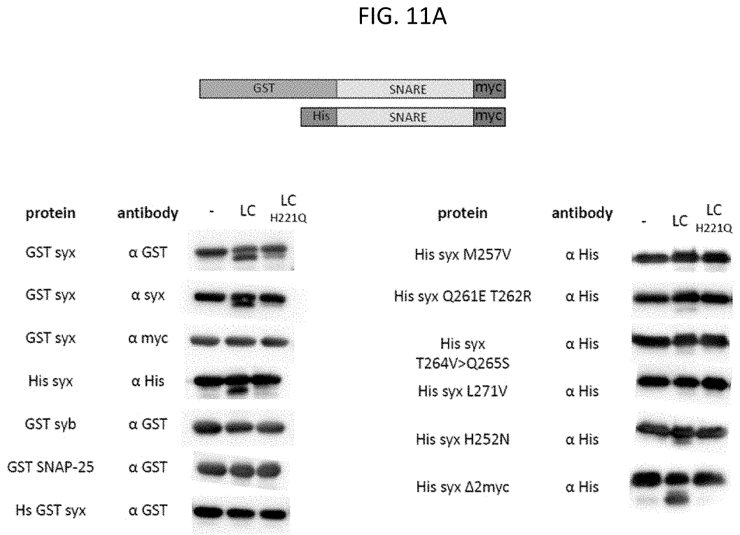

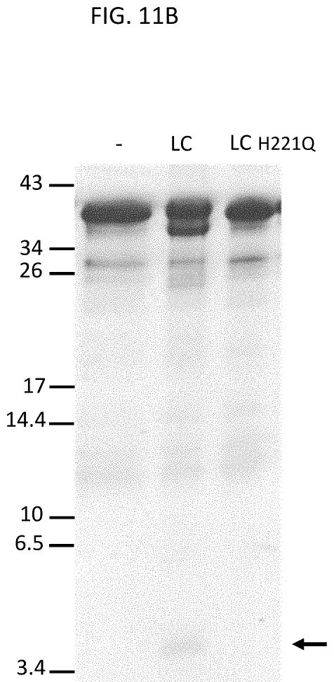

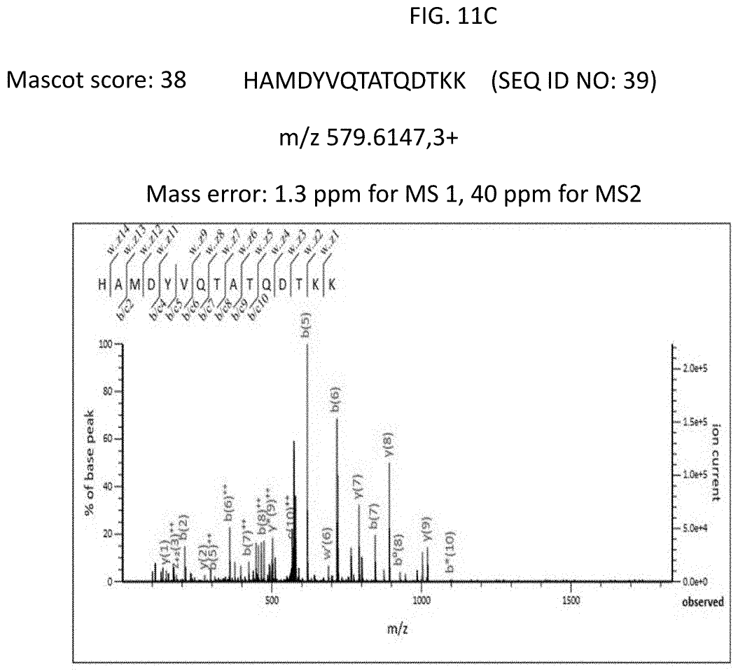

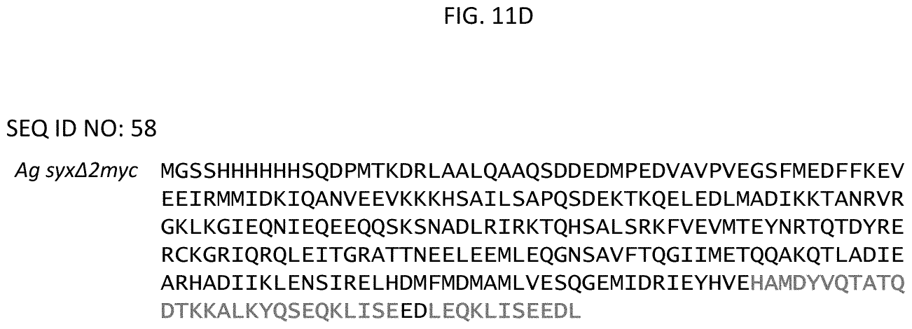

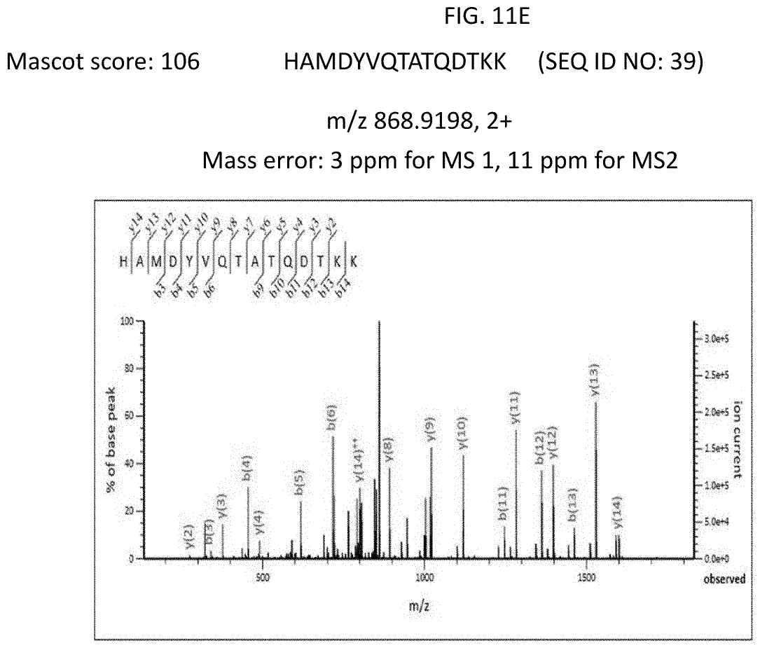

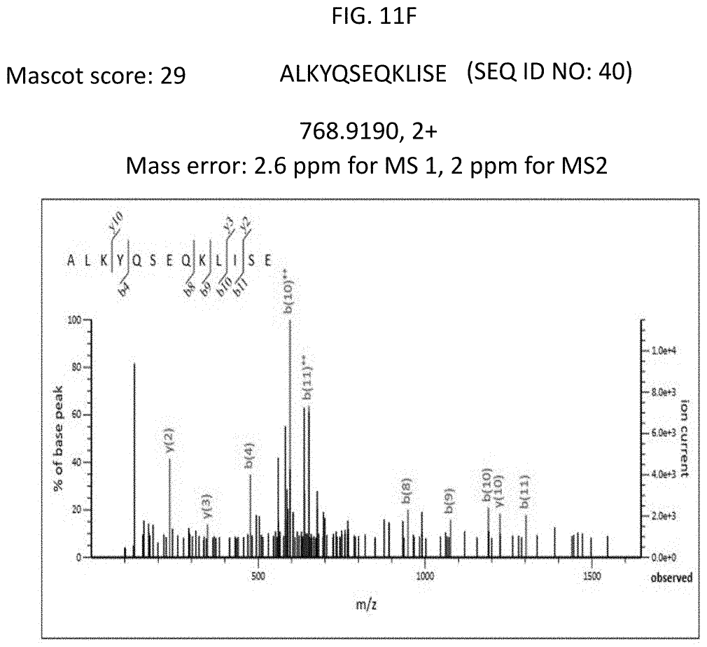

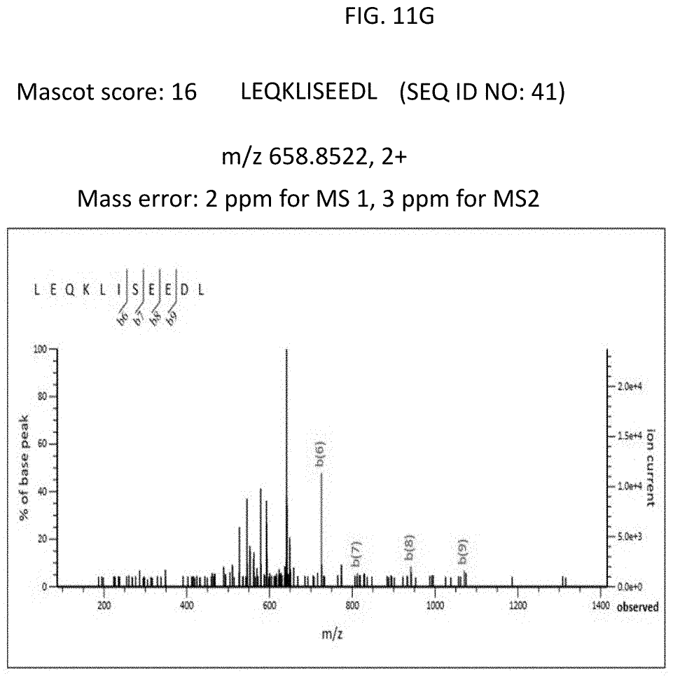

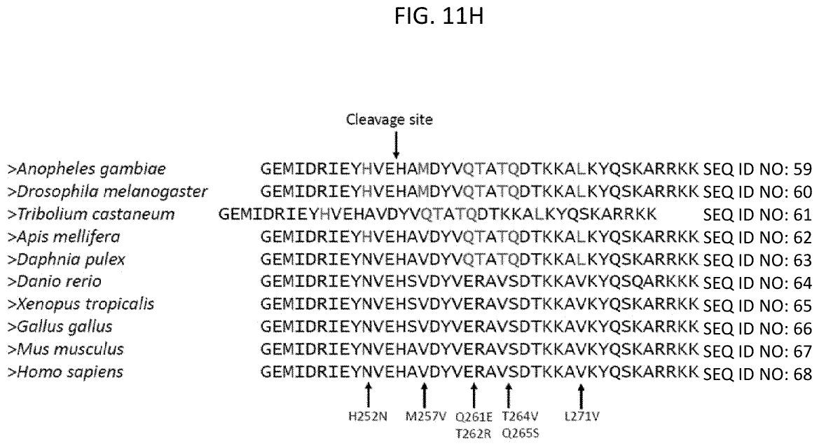

The metalloprotease activity of the LC of CNTs is specific for one of the three neuronal SNARE proteins in mammals. These neuronal proteins play a key role in the fusion of neurotransmitter-carrying vesicles to the plasma membrane thereby blocking neuroexocytosis. To determine if CMP1 exerts its action cleaving one of these SNARE protein homologs in mosquitoes, fragments of recombinant An. gambiae syntaxin1A, VAMP-2 and SNAP-25 were prepared and incubated with recombinant CMP1 LC. With reference to FIG. 11D it was observed that CMP1 LC cleaves the mosquito syntaxin resulting in a band of lower molecular weight that corresponds to cleavage of C-terminus of syntaxin, and no cleavage was observed by the catalytically inactive CMP1 LC E209Q mutant. Additionally, CMP1 LC was not able to cleave the recombinant human syntaxin1A (FIG. 11A) the C terminus of which is identical to its homolog in mouse and hence consistent with the lack of toxicity of CMP1 to mice by injection.

To determine CMP1 cleavage site, CMP1 LC and the mosquito syntaxin mixture were subjected to peptide purification and UPLC-MS/MS after incubation, with the aim of analyzing the peptide of around 4.5 KDa released from the cleavage as shown in FIG. 11B. Additionally, with reference to FIG. 11C, a unique peptide in syntaxin and CMP1 LC sample HAMDYVQTATQDTKK (SEQ ID NO: 39) was detected. Since the C terminus of syntaxin has a fragment rich in positive charges which makes it difficult to be detected by mass spectrometry, a syntaxin mutant where this region was deleted was created and similarly analyzed as shown in FIG. 4C. With reference to FIGS. 11D-11G, more peptides were detected and the region of C terminus of syntaxin was almost covered from H255 (FIGS. 11D-11G). As shown in FIG. 11H, CMP1 LC cleaves syntaxin between E254-H255 releasing a peptide that matches the observed size.

With reference to FIG. 11H, CMP1 cleavage site is conserved in human and mosquito syntaxin, despite the fact that the toxin is not able to cleave the human one. However, a region closer to the C terminus shows amino acid differences between human and mosquito syntaxins that could potentially influence the ability of CMP1 to accommodate syntaxin in the active site. To test this hypothesis, Anopheles syntaxin single and double mutants were prepared in which Anopheles amino acids in this region were switched to the corresponding amino acid residues in human syntaxin (FIG. 11H) and incubated with CMP1 LC. Syntaxin mutants were cleaved with less efficiency than non-mutated syntaxin and the L271V mutant completely abolished cleavage (FIG. 11A).

### Example 6. Materials and Methods

Insects. An. coluzzi, An. stephensi and Ae. aegypti mosquito larvae were reared at 28° C. with a photoperiod of 16:8 hours light/darkness in distilled water and fed with 1:4 yeast/koi fish food.

Bacterial strains and culture conditions. Clostridium bifermentans (ATCC) was used as the wild-type reference strain, and C. bifermentans subsp. malaysia and C. bifermentans subsp. paraiba was from the collection of the Institute for Medical Research, Malaysia as described in Lee and Seleena, 1990, Trop. Biomed. 7:103-106, the entire content of which is incorporated herein by reference. Bacteria was grown in liquid tryptone-yeast extract-glucose (TYG) medium at 30° C. under anaerobic conditions using BD GasPakEZ (Becton-Dickinson Microbiology).

Bacillus thuringiensis israelensis 4Q5 strain was grown overnight at 30° C. in sporulation media (0.8% Nutrient broth, 1 mM MgSO4, 13 mM KCl, 10 μM MnCl2, 0.5 mM CaCl2)) with shaking until complete autolysis.

Toxicity assays. Different volumes of Cbm or Bti whole bacterial culture were tested at room temperature in 100 ml water cups containing 20 third instar mosquito larvae. Bioassays were repeated at least 3 times, and LC₅₀ (the lethal concentration required to kill 50% of a population) were determined by probit analysis (USDA) and plotted using the Origin program (Origin Lab).

To test the constructs, Bacillus thuringiensis 4Q7 transformed strain was grown overnight at 30° C. in sporulation media with 50 μg/ml erythromycin and a 100× dilution in bioassay water cups was used.

Cb malaysia mutagenesis. A Cb malaysia overnight culture was diluted 1:30 in TYG media and grown for 6 hours in anaerobic conditions. The cells were exposed to a 137-Cesium source (J.L. Shepherd and Associates) for 6 minutes. Irradiated cells were diluted 1:100 and grown overnight at 30° C. in anaerobic conditions on TYG plates. Individual cells were selected and grown in liquid TYG for toxicity screening. Screening was performed using 3 Aedes 2nd instar larvae in 1 ml water in 24-well polystyrene plates and toxicity was recorded after 24 h. The mutants were then bioassayed with An. stephensi larvae.

PAGE and immunoblotting. Proteins were separated in a SDS-PAGE or native gel, transferred onto a PVDF membrane (Immobilon P, Millipore) and immunodetected as described in Qureshi et al., 2014, supra. Rabbit antibodies against the CMP1 peptide in the heavy chain (GFENIDFSEPEIRY) (SEQ ID NO: 42) was produced through commercial vendors.

Genomic DNA isolation. For genome sequencing, total Cb malaysia, Cb paraiba, and Cb DNA were isolated using phenol-chloroform extraction protocol and CbmΔ109 was isolated using DNeasy blood and tissue kit (Qiagen) from fresh overnight cultures. Quantity and quality of the DNA were measured spectrophotometrically (Nanodrop 2000, Thermo Scientific).

Proteomic analysis. Cb malaysia and CbmΔ109 proteins present in the culture supernatant were acid precipitated adding H2SO4 drop-wise to pH 3.5 according to Lin et al., 2015, supra. Precipitated proteins were extracted in agitation for 2 hours in 0.1M sodium citrate buffer pH 5.5 and analyzed in native protein acrylamide gels. Protein lanes were then excised from the gel and analyzed by mass spectrometry (LTQ Orbitrap Fusion MS coupled to 2-dimension nano-UPLC) at the Proteomics Core facility at the University of California, Riverside. Protein searches were performed against Cb malaysia genome predicted protein database.

For analysis of the cleavage site, cleavage assay mixtures after incubation were peptide purified using Sep-Pak cartridges (Waters) and analyzed by mass spectrometry as described above.

Larvae injection. Forth instar larvae were kept on ice and then injected between the head and the thorax on a petri dish using 3.5″ Drummond capillary tubes and a Nanoject II auto-nanoliter injector (Drummond Scientific). Injected larvae were transferred to water cups and kept for 24 hours under standard rearing conditions.

Plasmid construction, protein expression and purification. The cmp1 gene was commercially synthesized (GenScript) using B. thuringiensis codon optimization. The Ntnh-orfX1-orfX2-orfX3-cmp1 genes were amplified from Cb malaysia whole DNA preparation using Platinum Taq high fidelity polymerase (Thermo Fisher) and primers 1 and 2 (Table 1) in an automated thermocycler (C 1000 Touch, BioRad). Individual ntnh and cmp1 genes were amplified similarly using primers 3, 4 and 5, 6 respectively to produce constructs NTNH and NTNH-CMP1. PCR products were separated in 1% agarose gels and subsequently cut and purified using Wizard SV gel and PCR purification kits (Promega, Madison, Wis.). Sequencing of purified DNA products was performed by the Genomics Core facility at the University of California, Riverside. The full cmp1 operon, ntnh, cmp1 and ntnh-cmp1 constructs were first subcloned into pCR2.1 TOPO TA vector (Thermo Fisher) and then cloned into pHT315 vector (as described in Arantes and Lereclus, 1991, Gene 108:115-119, the entire content of which is incorporated herein by reference) under a Cyt1A promoter as described in Qureshi et al., 2014, supra for B. thuringiensis expression.

The constructs in pHT315 were used to transform B. thuringiensis subsp. israelensis 4Q7 cells (Bacillus Stock Center, Ohio State University, Columbus, Ohio).

CMP1, CMP1 catalytically inactive E209Q mutant, CMP1 HC mutants, CMP1 LC, CMP1 HC and SNARE proteins were purified from E. coli. CMP1 was commercially synthesized E. coli codon optimized and cloned in pQE-30 vector (Qiagen). Fragments of CMP1 HC containing the desired mutations were individually synthesized between restriction sites RsrII and HindIII, and were inserted in CMP1 to produce CMP1HC mutants. Catalytically inactive E209Q mutant was created by nested PCR using primers 7, 8, 9 and 10. CMP1 HC was amplified from CMP1 gene using primers 11 and 12 and cloned in pET duet 1. CMP1 LC was amplified from CMP1 gene using primers 13 and 14 and cloned in RSF duet 1. DNA sequence encoding fragments of SNARE proteins (A. gambiae VAMP-2 amino acids 1-99, syntaxin 1-268, SNAP-25 1-213, and Human VAMP-2 1-93 and syntaxin 1-266) were commercially synthesized codon optimized for E. coli expression with a myc tag added in C terminus (GenScript, Piscataway N.J.) and cloned in pGEX-6P vector. A. gambiae syntaxin with a His tag was amplified using primers 15 and 16 (Table 1) from synthesized syntaxin fragment and cloned in pET duet 1. Syntaxin mutants were produced by nested PCR, inserting the desired mutations in primers 17-28 (Table 1).

TABLE 1

| SEQ ID | Primer | Use | Sequence |
|---|---|---|---|
| 12 | 1 | CMP | GGCGCGCCATGGACATAATTGACAATGTAG |
| | | operon Fw | |
| 13 | 2 | CMP | CTCGAGCTATTCCTTCCATCCTTCATC |
| | | operon Rv | |
| 14 | 3 | NTNH Fw | CCCGGGATCCAATAATAGAAGGATATCAAAT |
| 15 | 4 | NTNH Rv | GCGGCCGCCCATTCATCGAAACATTCCCATCAT |
| 16 | 5 | CMP1 Fw | CTCGAGATATTTATTATAGATACCTTAAAGG |
| 17 | 6 | CMP1 Rv | CCACTTAATTGGTCAAATAACTATTCTTAATATGCTA |
| 18 | 7 | E209Q Fw | CGGCATCGAGCCTGACGCACCAACTGATCCATGCTCTGCAC |
| | | nested | |
| 19 | 8 | E209Q Rv | GTGCAGAGCATGGATCAGTTGGTGCGTCAGGCTCGATGCCG |
| | | nested | |
| 20 | 9 | CMP1/ | GGATCCCTGCAAATCCGTGTCTTTAACTATAACG |
| | | E209Q Fw | |
| 21 | 10 | CMP1/ | GGGCCCACATACGGGATAATCCAAGAGATGTC |
| | | E209Q Rv | |
| 22 | 11 | CMP1 Hc | GGATCCGAATGCCCTGATCGATCGCCTGGGTA |
| | | Fw | |
| 23 | 12 | CMP1 Hc | AAGCTTTCATTCTTTCCAACCTTCATCTTCC |
| | | Rv | |
| 24 | 13 | CMP1 LC | CCATGGACTACAAAGACGATGACGACAAGCTGCAAATCCGTGTCTT |
| | | Fw | TAACTATAACG |
| 25 | 14 | CMP1 LC | AAGCTTTCACAGTTTAACTTTTTTCGAGATCAG |
| | | Rv | |
| 26 | 15 | His syx Fw | CGGGATCCGATGACGAAGGACAGATTAGCAGCCCT |
| 27 | 16 | His syx Rv | GGCGCGCCTTACAGGTCTTCTTCAGAG |
| 28 | 17 | H252N Fw | GATTGATCGTATAGAATATAACGTCGAACATGCAATGG |
| 29 | 18 | H252N Rv | CCATTGCATGTTCGACGTTATATTCTATACGATCAATC |
| 30 | 19 | L271V Fw | CAAGACACAAAGAAAGCGGTCAAATATCAAAGCAAAGC |
| 31 | 20 | L271V Rv | GCTTTGCTTTGATATTTGACCGCTTTCTTTGTGTCTTG |
| 32 | 21 | T264V | GATTATGTTCAAACAGCGGTGTCTGACACAAAGAAAGCGC |
| | | Q265S Fw | |
| 33 | 22 | T264V | GCGCTTTCTTTGTGTCAGACACCGCTGTTTGAACATAATC |
| | | Q265S Rv | |
| 34 | 23 | Q261E | CAATGGATTATGTTGAAAGAGCGACACAAGACACAAAG |
| | | T262R Fw | |
| 35 | 24 | Q261E | CTTTGTGTCTTGTGTCGCTCTTTCAACATAATCCATTG |
| | | T262R Rv | |
| 36 | 25 | M257V Fw | CACGTCGAACATGCAGTGGATTATGTTCAAACAGCGAC |
| 37 | 26 | M257V Rv | GTCGCTGTTTGAACATAATCCACTGCATGTTCGACGTG |
| 38 | 27 | syx Δ2myc | GTTCCAGGTCTTCTTCAGAGATCAGTTTCTGTTCGCTTTGATATTTAA |
| | | 1 | GCGCTTTCTTTG |

BL21(DE3)pLysS chemically competent E. coli cells (Agilent) were transformed with genes cloned in vectors pGEX-6P, pET duet 1 and RSF duet 1 according to the manufacturer's protocol. Chemically competent M15 cells (Qiagen) were used for transformation of genes cloned in pQE-30. Cells were induced by adding 1 mM IPTG, grown in LB medium for 4 hours at 25° C. and harvested by centrifugation. Cell lysis was produced in 50 mM Tris, 300 mM NaCl, 1 mM DTT, 0.1% glycerol, 500 μg/ml lysozyme, pH 7.4 and sonicated for 3 min. CMP1 HC, CMP1, CMP1 mutants and syntaxin and syntaxin mutants with a His tag were purified from the lysate supernatant using Ni NTA agarose beads (Qiagen). LC was purified using Flag tag affinity gel (Biolegend) and the SNARE proteins with a GST tag were purified using GST SpinTrap columns (GE Healthcare).

Cleavage assays. Recombinant A. gambiae synaptobrevin, syntaxin, syntaxin mutants, SNAP-25 and Human syntaxin (2 ug) were incubated in 50 mM NaH2PO4 buffer pH 6.2 with 500 ng of LC, catalytically inactive E209Q LC or commercially available nicked BoNT/B (List Biological Laboratories, Campbell Calif.) for 3 hours at 30° C. Samples were analyzed by SDS-PAGE and western blot and immunodetected using GST tag antibody (GE Healthcare), His tag antibody (Genscript) Drosophila syntaxin antibody (Developmental Studies Hybridoma Bank, University of Iowa) or myc tag antibody (Cell Signaling).

### Example 7. SEQ ID NOS: 1-11

CMP1 protein sequence
(SEQ ID NO: 1)
MLQIRVFNYNDPIDGENIVELRYHNRSPVKAFQIVDGIWIIPERYNFTNDTKKVP
DDRALTILEDEVFAVRENDYLTTDVNEKNSFLNNITKLFKRINSSNIGNQLLNYISTSVPYP
VVSTNSIKARDYNTIKFDSIDGRRITKSANVLIYGPSMKNLLDKQTRAINGEEAKNGIGCLS
DIIFSPNYLSVQTVSSSRFVEDPASSLTHELIHALHNLYGIQYPGEEKFKFGGFIDKLLGTR
ECIDYEEVLTYGGKDSEIIRKKIDKSLYPDDFVNKYGEMYKRIKGSNPYYPDEKKLKQSFL
NRMNPFDQNGTFDTKEFKNHLMDLWFGLNESEFAKEKKILVRKHYITKQINPKYTELTND
VYTEDKGFVNGQSIDNQNFKIIDDLISKKVKLCSITSKNRVNICIDVNKEDLYFISDKEGFEN
IDFSEPEIRYDSNVTTATTSSFTDHFLVNRTFNDSDRFPPVELEYAIEPAEIVDNTIMPDIDQ
KSEISLDNLTTFHYLNAQKMDLGFDSSKEQLKMVTSIEESLLDSKKVYTPFTRTAHSVNER
ISGIAESYLFYQWLKTVINDFTDELNQKSNTDKVADISWIIPYVGPALNIGLDLSHGDFTKA
FEDLGVSILFAIAPEFATISLVALSIYENIEEDSQKEKVINKVENTLARRIEKWHQVYAFMVA
QWWGMVHTQIDTRIHQMYESLSHQIIAIKANMEYQLSHYKGPDNDKLLLKDYIYEAEIALN
TSANRAMKNIERFMIESSISYLKNNLIPSVVENLKKFDADTKKNLDQFIDKNSSVLGSDLHI
LKSQVDLELNPTTKVAFNIQSIPDFDINALIDRLGIQLKDNLVFSLGVESDKIKDLSGNNTNL
EVKTGVQIVDGRDSKTIRLNSNENSSIIVQKNESINFSYFSDFTISFWIRVPRLNKNDFIDLG
IEYDLVNNMDNQGWKISLKDGNLVWRMKDRFGKIIDIITSLTFSNSFIDKYISSNIWRHITIT
VNQLKDCTLYINGDKIDSKSINELRGIDNNSPIIFKLEGNRNKNQFIRLDQFNIYQRALNESE
VEMLFNSYFNSNILRDFWGEPLEYNKSYYMINQAILGGPLRSTYKSWYGEYYPYISRMRT
FNVSSFILIPYLYHKGSDVEKVKIINKNNVDKYVRKNDVADVKFENYGNLILTLPMYSKIKE
RYMVLNEGRNGDLKLIQLQSNDKYYCQIRIFEMYRNGLLSIADDENWLYSSGWYLYSSG
WYLDNYKTLDLKKHTKTNWYFVSEDEGWKE
CMP1 DNA sequence
(SEQ ID NO: 2)
ATGCTACAAATAAGAGTTTTTAATTATAATGATCCAATTGATGGAGAAAATAT
CGTGGAGTTAAGATACCATAACAGGAGCCCTGTAAAAGCATTTCAAATAGTAGATGGT
ATATGGATAATTCCAGAAAGATATAACTTTACAAACGATACAAAAAAAGTTCCAGACG
ATCGAGCTCTTACTATTCTGGAAGATGAAGTTTTTGCTGTTCGCGAAAATGACTATTTA
ACAACAGATGTTAATGAAAAAAATTCCTTTTTAAATAATATTACTAAGCTTTTTAAGCGT
ATTAATTCAAGTAACATTGGTAATCAGTTACTTAATTATATTTCAACAAGCGTCCCATA
TCCAGTTGTGAGTACAAATTCAATAAAGGCTAGAGACTATAATACAATTAAATTTGATT
CAATTGATGGGCGAAGAATTACAAAATCTGCAAATGTACTTATCTACGGACCAAGTAT
GAAAAATTTACTAGATAAACAAACAAGGGCTATCAATGGGGAAGAAGCAAAAAATGGT
ATAGGATGTTTAAGTGATATTATTTTTTCTCCAAATTACTTATCTGTCCAAACTGTTTCT
TCAAGTAGGTTTGTTGAAGATCCTGCATCATCACTTACACATGAACTTATCCATGCCT
TACATAATTTATATGGAATACAATATCCTGGAGAAGAAAAATTTAAATTTGGAGGATTT
ATTGATAAACTATTAGGAACTAGAGAATGCATAGATTATGAGGAAGTCTTAACATATG
GAGGAAAAGATTCCGAAATTATAAGAAAGAAAATTGATAAGTCCTTATATCCTGATGA
TTTTGTAAATAAGTATGGTGAAATGTATAAGCGTATAAAAGGATCTAATCCTTATTATC
CCGACGAAAAAAAATTAAAACAAAGTTTTTTAAACAGAATGAATCCATTTGATCAAAAT
GGAACTTTTGATACTAAAGAATTTAAAAATCATCTTATGGATTTATGGTTTGGGTTAAA
TGAGAGTGAATTTGCTAAAGAAAAGAAGATTTTAGTCAGAAAGCACTATATAACAAAG
CAAATTAATCCTAAATACACAGAACTTACTAATGATGTATATACTGAAGATAAAGGCTT
TGTAAATGGTCAATCTATAGACAATCAAAATTTTAAAATAATTGATGATTTAATATCAAA
AAAAGTAAAACTATGTTCTATAACATCTAAAAATCGAGTAAATATTTGTATAGACGTTA
ATAAAGAAGATTTATATTTCATAAGTGATAAAGAAGGTTTTGAAAATATAGATTTTTCC
GAGCCGGAAATTAGATATGATAGTAATGTAACTACAGCAACTACCTCTTCTTTTACAG
ACCATTTTTTAGTAAATAGAACTTTTAACGATAGTGATAGATTTCCACCTGTAGAATTA
GAATATGCTATCGAACCAGCTGAAATAGTTGATAACACTATAATGCCAGATATTGATC
AAAAAAGCGAAATATCTCTCGATAACTTAACGACCTTTCACTATTTAAATGCTCAAAAA
ATGGATTTGGGATTTGATTCATCAAAAGAACAGTTAAAGATGGTTACATCAATAGAGG
AATCATTATTAGATTCAAAAAAGGTATACACACCATTTACGAGAACTGCACATAGTGTA
AATGAACGTATATCTGGAATAGCGGAAAGTTACTTATTTTATCAATGGTTAAAAACTGT
TATAAATGATTTTACAGATGAATTAAACCAAAAGAGTAATACTGACAAAGTTGCTGATA
TTTCTTGGATTATACCCTATGTTGGACCTGCTTTAAATATTGGCCTTGATTTATCTCAT
GGAGATTTTACTAAAGCTTTTGAAGATTTAGGGGTTTCTATTTTATTTGCTATTGCTCC
AGAATTTGCAACTATAAGTCTTGTAGCTCTTTCAATATATGAAAATATAGAAGAGGATT
CACAAAAAGAAAAAGTAATTAATAAAGTAGAAAATACATTAGCAAGGAGAATAGAAAA
ATGGCACCAAGTTTATGCTTTCATGGTGGCTCAGTGGTGGGGTATGGTTCATACTCA
GATAGACACTAGAATTCATCAAATGTATGAATCACTTTCTCATCAAATTATAGCAATTA
AAGCTAATATGGAGTATCAGTTATCTCATTATAAAGGCCCTGATAATGATAAACTTCTA
TTAAAGGATTATATATATGAGGCTGAAATAGCTCTTAACACTTCAGCAAATCGAGCAA
TGAAAAATATTGAAAGATTTATGATTGAAAGCTCTATTTCATACTTAAAAAATAATCTAA
TTCCCAGTGTAGTAGAAAATTTAAAAAAATTTGATGCTGATACAAAAAAGAATTTAGAT
CAATTTATTGATAAAAATTCCTCAGTATTAGGATCTGATTTACATATATTAAAGTCTCAA
GTAGATTTAGAACTTAATCCAACTACTAAGGTAGCCTTTAATATTCAAAGTATTCCAGA
TTTTGATATAAATGCATTGATAGACAGATTAGGTATTCAATTAAAAGATAACTTAGTATT
TAGTTTAGGAGTGGAATCTGATAAAATAAAAGATCTATCTGGGAATAATACAAACCTA
GAAGTTAAAACAGGTGTCCAAATAGTAGATGGACGAGATAGTAAGACTATACGTTTAA
ATTCAAATGAAAATTCAAGTATTATAGTTCAGAAAAATGAAAGTATAAACTTCTCATATT
TTAGTGACTTTACCATAAGTTTTTGGATAAGAGTTCCAAGACTTAATAAAAATGATTTT
ATAGACTTAGGAATTGAATATGACTTAGTAAATAATATGGATAATCAAGGATGGAAAAT
TTCGCTTAAGGATGGGAATTTAGTATGGAGAATGAAAGATAGATTTGGAAAAATAATA
GATATTATTACGTCTTTAACCTTTAGTAATAGCTTTATAGATAAATATATATCCAGTAAT
ATATGGAGACATATAACTATTACAGTTAACCAATTAAAAGATTGTACTTTATATATAAAT
GGAGATAAAATAGATAGTAAATCAATTAACGAATTAAGAGGTATCGATAATAATTCTCC
AATAATATTCAAGTTAGAAGGGAATAGAAATAAAAATCAATTTATACGCTTAGATCAGT
TTAATATTTATCAAAGGGCTTTAAATGAAAGTGAAGTTGAAATGTTATTTAATAGTTATT
TTAATTCAAATATATTAAGAGATTTTTGGGGAGAACCTTTAGAGTATAATAAGAGTTAC
TATATGATAAATCAAGCAATATTAGGTGGACCCCTTAGAAGCACATATAAGTCATGGT
ATGGAGAGTATTACCCTTATATATCTAGAATGAGGACGTTTAATGTTTCATCATTTATT
TTAATTCCTTACCTATATCATAAAGGATCAGATGTAGAAAAGGTAAAAATAATAAATAA
AAACAACGTGGATAAATATGTAAGAAAAAATGATGTAGCAGATGTTAAATTTGAAAATT
ATGGTAATTTAATACTTACGTTACCTATGTACAGTAAAATCAAAGAGAGATATATGGTA
TTAAACGAGGGTAGAAACGGCGATTTAAAGTTAATTCAATTACAAAGTAACGATAAAT
ACTATTGTCAAATACGAATATTTGAAATGTACAGAAATGGGTTGCTGTCAATTGCAGA
CGATGAAAACTGGTTATACTCTAGTGGCTGGTATTTATACTCTAGTGGCTGGTATTTA
GATAATTATAAAACTTTGGATTTAAAAAAACATACAAAAACTAATTGGTATTTTGTTAGT
GAAGATGAAGGATGGAAGGAATAG
NTNH protein sequence
(SEQ ID NO: 3)
MDIIDNVDITLPENGEDIVIVGGRRYDYNGDLAKFKAFKVAKHIWVVPGRYYGE
KLDIQDGEKINGGIYDKDFLSQNQEKQEFMDGVILLLKRINNTLEGKRLLSLITSAVPFPNE
DDGIYKQNNFILSDKTFKAYTSNIIIFGPGANLVENKVIAFNSGDAENGLGTISEICFQPLLT
YKFGDYFQDPALDLLKCLIKSLYYLYGIKVPEDFTLPYRLTNNPDKTEYSQVNMEDLLISG
GDDLNAAGQRPYWLWNNYFIDAKDKFDKYKEIYENQMKLDPNLEINLSNHLEQKFNINIS
ELWSLNISNFARTFNLKSPRSFYKALKYYYRKKYYKIHYNEIFGTNYNIYGFIDGQVNASLK
ETDLNIINKPQQIINLIDNNNILLIKSYIYDDELNKIDYNFYNNYEIPYNYGNSFKIPNITGILLP
SVNYELIDKIPKIAEIKPYIKDSTPLPDSEKTPIPKELNVGIPLPIHYLDSQIYKGDEDKDFILS
PDFLKVVSTKDKSLVYSFLPNIVSYFDGYDKTKISTDKKYYLWIREVLNNYSIDITRTENIIGI
FGVDEIVPWMGRALNILNTENTFETELRKNGLKALLSKDLNVIFPKTKVDPIPTDNPPLTIE
KIDEKLSDIYIKNKFFLIKNYYITIQQWWICCYSQFLNLSYMCREAIINQQNLIEKIILNQLSYL
ARETSINIETLYILSVTTEKTIEDLREISQKSMNNICNFFERASVSIFHTDIYNKFIDHMKYIVD
DANTKIINYINSNSNITQEEKNYLINKYMLTEEDFNFFNFDKLINLFNSKIQLTIKNEKPEYNL
LLSINQNESNENITDISGNNVKISYSNNINILDGRNEQAIYLDNDSQYVDFKSKNFENGVTN
NFTISFWMRTLEKVDTNSTLLTSKLNENSAGWQLDLRRNGLVWSMKDHNKNEINIYLNDF
LDISWHYIVVSVNRLTNILTVYIDGELSVNRNIEEIYNLYSDVGTIKLQASGSKVRIESFSILN
RDIQRDEVSNRYINYIDNVNLRNIYGERLEYNKEYEVSNYVYPRNLLYKVNDIYLAIERGSN
SSNRFKLILININEDKKFVQQKDIVIIKDVTQNKYLGISEDSNKIKLVDRNNALELILDNHLLN
PNYTTFSTKQEEYLRLSNIDGIYNWVIKDVSRLNDIYSVVTLI
NTNH DNA sequence
(SEQ ID NO: 4)
ATGGACATAATTGACAATGTAGATATAACATTACCTGAAAATGGTGAAGATA
TTGTAATCGTAGGAGGAAGAAGATATGATTATAATGGAGACTTAGCAAAATTTAAAGC
TTTTAAAGTGGCTAAGCATATTTGGGTGGTTCCAGGTAGATATTATGGTGAAAAATTA
GATATACAAGATGGTGAAAAAATTAATGGAGGAATTTATGACAAAGATTTTTTATCTCA
GAATCAAGAAAAACAAGAATTTATGGATGGAGTTATACTCTTATTAAAAAGAATCAATA
ATACGTTAGAAGGAAAAAGATTATTATCGCTTATAACATCCGCTGTACCTTTTCCTAAC
GAAGATGATGGAATATATAAACAAAATAACTTTATACTTTCTGATAAAACGTTTAAAGC
GTATACTTCAAATATTATTATTTTTGGTCCTGGAGCAAACTTGGTAGAGAATAAAGTTA
TTGCATTTAATAGTGGTGATGCTGAAAATGGACTTGGAACAATATCAGAAATTTGTTTT
CAACCGCTTTTAACTTATAAATTTGGAGATTATTTTCAGGACCCTGCACTAGATTTATT
AAAGTGTTTAATAAAATCCTTATATTATTTGTATGGAATTAAAGTTCCAGAAGATTTTAC
TTTACCGTATAGGTTGACGAATAATCCAGATAAGACAGAATATTCTCAGGTCAATATG
GAAGATTTATTAATATCAGGTGGTGATGATCTTAATGCTGCAGGGCAGAGACCATATT
GGCTATGGAATAATTATTTTATAGACGCAAAGGATAAATTTGATAAATATAAAGAAATT
TACGAAAACCAAATGAAACTGGATCCTAATCTAGAAATTAATCTTTCAAATCATTTAGA
GCAAAAATTTAATATAAACATATCTGAATTATGGAGCTTAAACATATCTAATTTTGCAA
GAACATTTAATTTAAAATCACCTAGAAGTTTTTATAAAGCACTTAAATATTATTATAGAA
AAAAATATTATAAGATACATTATAATGAAATATTTGGAACAAATTATAATATATATGGAT
TTATAGATGGACAAGTTAATGCATCACTAAAAGAAACTGATTTAAATATTATAAATAAA
CCACAGCAGATTATTAACCTTATTGATAATAACAATATATTATTAATAAAGTCCTATATA
TATGACGATGAATTAAATAAAATAGATTATAATTTTTATAATAATTATGAAATCCCTTAT
AACTATGGAAATTCTTTTAAAATACCTAATATAACGGGAATACTTTTACCTAGCGTAAA
TTATGAATTAATTGATAAAATACCAAAAATTGCTGAAATTAAACCTTATATTAAAGACTC
AACACCATTACCAGATTCTGAAAAAACGCCTATTCCTAAAGAGTTAAATGTAGGAATT
CCATTACCTATTCATTATTTGGATTCACAAATTTATAAAGGAGATGAAGATAAAGATTT
TATATTATCTCCTGACTTTCTAAAGGTTGTGTCCACCAAAGATAAATCTCTAGTATATA
GCTTTTTACCCAATATTGTTTCATATTTTGATGGATATGATAAAACAAAAATTTCTACTG
ACAAAAAATATTATTTATGGATAAGGGAAGTTTTAAATAATTATTCAATAGATATAACTA
GAACTGAAAATATAATTGGTATTTTTGGAGTAGATGAGATAGTTCCTTGGATGGGAAG
GGCCTTGAATATCTTAAATACAGAAAATACTTTTGAAACTGAACTTAGAAAAAATGGCT
TAAAAGCTTTGCTTTCTAAAGATTTAAACGTTATTTTCCCAAAAACAAAAGTGGATCCA
ATACCTACAGATAATCCTCCCCTTACAATAGAAAAAATAGATGAAAAACTTTCAGATAT
TTATATTAAAAATAAATTCTTTTTAATAAAAAATTACTACATAACTATACAGCAATGGTG
GATATGTTGCTATAGTCAATTTTTAAATCTTAGTTATATGTGTCGTGAAGCAATAATAA
ATCAACAAAATTTAATTGAAAAAATTATTTTAAATCAACTCAGCTATTTAGCTCGTGAG
ACAAGCATTAACATAGAAACGTTGTATATATTAAGTGTAACAACTGAAAAGACAATAGA
AGATTTAAGAGAAATATCACAAAAGTCAATGAATAATATATGCAATTTTTTTGAACGAG
CTAGTGTTTCAATATTCCATACTGATATTTACAATAAGTTTATTGATCATATGAAATATA
TAGTTGATGATGCAAATACTAAGATTATAAATTATATAAATTCTAATTCTAATATTACAC
AAGAAGAAAAAAATTACTTAATTAATAAATATATGCTAACAGAAGAAGATTTTAATTTTT
TCAATTTTGATAAATTAATAAATTTATTTAATTCTAAAATTCAACTCACAATTAAAAATGA
AAAGCCGGAATATAATTTATTACTATCTATAAATCAAAATGAGAGTAATGAGAATATTA
CCGATATATCAGGAAATAATGTAAAAATTAGTTATTCAAATAATATTAACATATTAGATG
GCAGAAATGAACAGGCAATATATTTAGATAATGATAGTCAATATGTTGACTTCAAATCT
AAAAATTTTGAAAATGGAGTAACTAATAATTTTACAATTAGTTTTTGGATGAGAACTTTA
GAGAAAGTAGACACAAATTCTACATTGTTAACATCTAAACTTAATGAGAATTCTGCAG
GATGGCAACTGGATTTAAGAAGAAATGGATTAGTTTGGAGTATGAAAGATCACAACAA
AAATGAAATAAATATTTATTTAAATGATTTTTTAGATATAAGTTGGCACTATATCGTTGT
TTCAGTTAATCGTTTAACAAATATATTAACTGTATATATAGATGGTGAGCTTAGTGTTA
ACAGAAATATTGAGGAAATATATAATCTATATTCAGATGTGGGGACAATTAAACTGCA
AGCAAGTGGATCTAAAGTTCGCATTGAATCTTTTTCGATTTTAAACAGAGACATTCAAA
GAGATGAGGTATCTAATAGATACATTAATTATATTGATAATGTAAATTTAAGGAATATA
TATGGGGAGAGATTAGAATACAACAAGGAATATGAAGTATCTAATTATGTTTATCCTA
GAAACTTACTATACAAGGTCAATGATATATATTTAGCTATTGAGAGAGGAAGCAACAG
TTCTAACAGGTTTAAATTAATATTAATAAATATAAATGAAGATAAAAAATTTGTACAGCA
AAAAGACATAGTTATTATTAAAGATGTCACTCAAAATAAATATTTAGGTATTTCAGAAG
ATAGTAATAAGATTAAGCTAGTAGATAGAAATAATGCTTTAGAGTTGATTCTAGATAAT
CATCTTCTTAATCCTAATTATACGACATTTTCTACTAAACAAGAAGAATATTTAAGACTA
TCTAATATAGATGGAATATATAACTGGGTGATAAAGGATGTATCGAGATTAAATGATAT
ATATTCTTGGACTTTAATATAA
OrfX1 protein sequence
(SEQ ID NO: 5)
MNREFPFHFNDGNVSMNGLFCLKKIKTQYHPNYDYFKIKFCEGFLSIKNKVKD
DLCEYDLKNIESVIALKREYSKENNLKNKESAIFMNIGNKGIHNKYDLYVVNVDINNILDEN
YMLKGILNDKLKILFLGNERKLLRIKN
OrfX1 DNA sequence
(SEQ ID NO: 6)
ATGAATAGGGAGTTTCCATTCCATTTTAATGATGGGAATGTTTCGATGAATG
GATTATTTTGTTTAAAGAAAATAAAAACGCAATATCATCCAAATTATGATTATTTCAAAA
TTAAATTCTGTGAAGGGTTTTTATCTATAAAGAATAAGGTTAAAGATGATTTGTGTGAA
TATGATTTGAAAAACATTGAATCCGTAATTGCATTAAAAAGAGAATATTCAAAAGAAAA
TAATTTAAAAAATAAAGAATCAGCAATTTTTATGAATATTGGGAATAAAGGGATTCATA
ATAAATATGATTTATATGTTGTAAATGTAGATATTAACAATATTTTAGATGAAAATTATA
TGTTAAAAGGAATATTAAATGATAAGCTAAAGATTCTTTTTTTAGGTAATGAAAGGAAG
TTATTAAGAATAAAAAATTAG
OrfX2 protein sequence
(SEQ ID NO: 7)
MSKKPLDFLRIYDWHKTEAMNKISKLDFERIIPKHFSKEIKNKHLSVKITGNWKI
WKLTDEGEGQYPIFKCIVEDGFLKIKNECGNKKYSLDNAWIKICTKIKYDNENGKDIYSIDE
KNLTLYSVNNSFNSKYKNNIVDAFLDNLLIACIEDNIKDLNKFFKLYKVKTAIKEDLSLLGWD
TGYSTSFTHVNKTIENQQNYPKQFKYESEGPYNIDISGEFDSWRLTTGSDGQNVNFICPI
KNGEFNFLGTEYKFSQGEQVNIQLKLKYLNIEEPTFEDSTSLNDGNQVDLIVKTDEDENE
NPPVTIIKVVLLGEIDAIGKMLLEGTFREWFNENIDAFKQIFSSFLLEDTSKNPDFQWLKPT
KAYYGVASAEPIDGKPDLDSSVFSVMSMVEDNKNDKPSHTVDGRILDAVNNESAFGIRTP
LFVKKWLIAGLEMMQIGKLEDFDLINNGMGFINNKKLLFGTFENADGEDVPAYVEKDNFR
LEITNNQLKIEITDIYWQQSRRLTGHVMYSQYFDLELRSGTDITGAEYKNILIPVENSEPTLV
VNISQDEFDIWGDIVGEIVGGIVVGIVTGYLGSILGKGVGKYLEKFLTKTSGGRWVLKMNK
EMYDYLNNLFKGDRRVFNEVAIDEIELISTLGTSQAISTIANTPTNFASKIWVNKSKFIGGLI
GGSVGSVIPSVIIKSIDAWDKQNYSVLPSINAFVASSVGSVKWPDTSEFKIESAELNGIFLL
GGKLERYEK
OrfX2 DNA sequence
(SEQ ID NO: 8)
ATGAGTAAAAAACCATTAGATTTTCTAAGAATTTATGATTGGCATAAAACTG
AAGCAATGAACAAAATTAGTAAACTAGATTTTGAAAGGATAATTCCTAAACATTTTTCA
AAAGAAATTAAAAATAAACACTTAAGTGTTAAAATTACTGGTAACTGGAAAATTTGGAA
GTTAACAGATGAAGGAGAAGGGCAATATCCTATTTTTAAATGCATAGTTGAAGATGGA
TTCTTAAAAATAAAAAATGAATGTGGAAATAAAAAATATTCACTAGATAATGCTTGGAT
AAAAATTTGTACAAAAATTAAATATGATAATGAAAATGGAAAAGATATCTATTCAATAG
ATGAAAAAAACTTAACATTGTACAGTGTTAATAATTCATTTAACTCAAAATATAAAAATA
ATATTGTAGATGCTTTTTTAGATAATTTATTAATAGCGTGTATTGAGGACAATATAAAA
GATTTAAATAAGTTTTTTAAGCTATATAAAGTTAAAACAGCAATAAAAGAAGATTTAAGT
CTCTTAGGATGGGATACAGGATACTCAACATCATTTACTCATGTAAATAAAACTATTGA
AAATCAACAGAATTATCCGAAGCAGTTTAAATATGAGTCTGAGGGTCCTTATAACATT
GATATATCTGGAGAATTTGATTCATGGAGATTAACTACTGGATCAGATGGTCAAAATG
TTAATTTTATTTGTCCAATTAAAAATGGTGAATTTAACTTTTTGGGAACCGAGTATAAAT
TTTCACAAGGTGAACAAGTTAATATACAACTTAAGTTAAAATATTTAAATATTGAAGAG
CCAACCTTTGAAGATTCAACTTCCTTAAATGATGGAAATCAGGTTGATTTAATTGTTAA
AACAGATGAAGACGAGAATGAAAATCCTCCGGTTACAATTATAAAAGTAGTTTTACTA
GGTGAAATTGACGCTATTGGTAAGATGCTTTTAGAGGGTACGTTTAGAGAGTGGTTTA
ATGAAAATATTGATGCATTTAAACAAATATTTTCTTCTTTCCTTTTAGAGGATACATCTA
AAAATCCAGATTTTCAGTGGTTAAAACCTACAAAGGCTTATTATGGAGTTGCAAGTGC
TGAACCAATAGACGGAAAGCCTGACTTAGATAGTAGTGTATTTTCTGTCATGTCTATG
GTAGAAGATAATAAAAATGATAAACCAAGTCATACAGTAGATGGTAGAATACTTGATG
CTGTTAATAATGAATCTGCATTTGGAATTAGAACCCCATTATTTGTTAAAAAATGGCTT
ATTGCCGGACTAGAAATGATGCAAATTGGAAAATTAGAAGATTTTGATTTAATAAATAA
CGGAATGGGATTTATTAATAACAAGAAACTTTTGTTTGGTACTTTTGAAAATGCTGATG
GTGAAGATGTACCTGCTTATGTAGAAAAAGATAATTTTAGATTAGAAATAACGAATAAT
CAACTAAAAATAGAAATAACAGATATATATTGGCAGCAATCAAGAAGATTAACAGGGC
ATGTAATGTATAGCCAATATTTTGATTTAGAATTAAGAAGCGGAACTGATATCACTGGA
GCAGAATATAAAAATATTTTAATTCCAGTAGAAAATTCAGAGCCAACATTGGTAGTAAA
CATTTCACAAGATGAATTTGATATTTGGGGAGATATTGTCGGTGAAATAGTTGGAGGT
ATAGTTGTGGGAATAGTCACAGGTTACTTAGGTAGCATTTTAGGCAAAGGAGTAGGA
AAATATTTAGAAAAATTCCTTACAAAAACATCTGGTGGAAGATGGGTATTAAAAATGAA
TAAAGAGATGTATGATTATTTAAATAATTTATTTAAAGGAGATAGAAGAGTTTTCAATG
AAGTTGCCATAGATGAAATAGAACTGATTTCAACATTAGGAACATCTCAAGCTATATC
AACAATTGCAAATACACCTACTAATTTTGCATCTAAAATATGGGTAAATAAATCAAAAT
TTATAGGTGGTTTAATTGGGGGGTCAGTAGGCTCAGTAATACCTAGCGTTATTATAAA
ATCAATAGACGCTTGGGATAAACAAAATTATTCTGTTCTTCCAAGTATAAATGCATTTG
TAGCTTCAAGTGTAGGTTCTGTAAAATGGCCGGATACCAGTGAATTCAAGATTGAATC
AGCTGAGCTTAACGGAATTTTTTTGTTAGGTGGAAAGCTAGAAAGATATGAAAAATAA
OrfX3 protein sequence
(SEQ ID NO: 9)
MIGKRQTSTLNWDTVFAVPISVVNKAIKDKKSSPENFEFEDSSGSKCKGDFGD
WQIITGGDGSNIRMKIPIYNFKAELVDDKYGIFNGNGGFESGEMNIQVKLKYFPHDKISKY
KDVELVDLKVRSESADPIDPVVVMLSLKNLNGFYFNFLNEFGEDLQDIIEMFFIELVKQWL
TENISLFNHIFSVVNLNLYIDQYSQWSWSRPSYVSYAYTDIEGDLDKSLLGVLCMTGGRN
PDLRQQKVDPHAVPESSQCGFLIYEERVLRDLLLPTLPMKFKNSTVEDYEVINASGESGQ
YQYILRLKKGRSVSLDRVEANGSKYDPYMTEMSISLSNDVLKLEATTETSVGMGGKVGC
DTINWYKLVLAKNGNGEQTISYEEVGEPTVINYVIKEGENWVWDVIAAIIAILATAVLAIFTG
GAAFFIGGIVIAIITGFIAKTPDIILNWNLETSPSIDMMLENSTSQIIWNARDIFELDYVALNGP
LQLGGELTV
OrfX3 DNA sequence
(SEQ ID NO: 10)
ATGATAGGAAAACGTCAAACAAGTACACTGAATTGGGATACAGTATTTGCT
GTTCCTATTAGTGTAGTAAATAAAGCGATAAAAGATAAAAAAAGTAGCCCTGAGAATT
TTGAATTTGAAGATTCATCTGGTAGTAAATGTAAAGGGGATTTTGGAGATTGGCAAAT
AATTACTGGTGGTGATGGAAGTAATATACGAATGAAAATTCCTATTTACAATTTTAAAG
CTGAACTGGTCGATGATAAATATGGAATTTTTAATGGAAACGGTGGATTTGAATCTGG
AGAAATGAATATTCAAGTTAAGCTTAAGTATTTTCCACATGATAAAATATCAAAATATAA
AGATGTTGAATTAGTTGATTTAAAAGTAAGATCAGAAAGTGCTGATCCAATTGATCCA
GTAGTAGTTATGCTCTCATTGAAGAATTTAAATGGGTTTTATTTTAATTTTTTAAATGAA
TTTGGTGAAGATTTACAAGATATTATAGAGATGTTTTTTATAGAGCTCGTTAAACAATG
GCTGACAGAAAATATTAGTTTATTTAACCATATTTTTAGTGTAGTAAACTTAAATTTATA
TATTGATCAATATTCTCAATGGTCATGGAGTAGGCCTTCATATGTTAGCTATGCTTATA
CAGATATAGAAGGTGATTTAGATAAAAGTCTATTAGGGGTTTTGTGTATGACAGGAGG
AAGAAATCCTGATCTTAGACAACAGAAGGTAGATCCTCATGCAGTACCAGAAAGTTCT
CAATGTGGATTTTTAATTTATGAAGAGAGGGTATTAAGAGATTTACTTTTACCAACTTT
ACCAATGAAATTTAAAAATTCAACAGTAGAAGATTATGAGGTAATTAATGCAAGCGGA
GAAAGTGGTCAGTATCAGTATATATTAAGATTAAAAAAAGGTAGGAGTGTTAGTTTAG
ACCGCGTTGAGGCTAATGGTTCTAAATATGATCCATATATGACTGAAATGAGTATTAG
TTTATCAAATGATGTATTAAAACTAGAAGCAACCACAGAAACTTCGGTAGGAATGGGA
GGAAAAGTTGGATGTGATACTATAAATTGGTATAAGTTAGTACTTGCAAAAAATGGAA
ATGGAGAACAAACTATATCATATGAAGAAGTTGGAGAACCTACAGTAATAAATTATGT
AATAAAAGAAGGCGAAAATTGGGTATGGGATGTAATCGCTGCAATCATAGCTATTCTA
GCAACAGCAGTATTGGCAATATTTACTGGAGGAGCAGCTTTTTTTATAGGTGGTATTG
TTATAGCTATAATAACAGGATTTATAGCTAAAACTCCAGATATAATTTTAAATTGGAAC
CTTGAAACTTCTCCAAGTATAGATATGATGTTAGAAAATTCTACTTCACAAATTATTTG
GAATGCTAGAGACATATTTGAACTAGATTATGTTGCTTTAAATGGACCACTGCAACTA
GGTGGAGAATTAACTGTTTAA
Cmp1 Operon DNA sequence
(SEQ ID NO: 11)
ATGGACATAATTGACAATGTAGATATAACATTACCTGAAAATGGTGAAGATA
TTGTAATCGTAGGAGGAAGAAGATATGATTATAATGGAGACTTAGCAAAATTTAAAGC
TTTTAAAGTGGCTAAGCATATTTGGGTGGTTCCAGGTAGATATTATGGTGAAAAATTA
GATATACAAGATGGTGAAAAAATTAATGGAGGAATTTATGACAAAGATTTTTTATCTCA
GAATCAAGAAAAACAAGAATTTATGGATGGAGTTATACTCTTATTAAAAAGAATCAATA
ATACGTTAGAAGGAAAAAGATTATTATCGCTTATAACATCCGCTGTACCTTTTCCTAAC
GAAGATGATGGAATATATAAACAAAATAACTTTATACTTTCTGATAAAACGTTTAAAGC
GTATACTTCAAATATTATTATTTTTGGTCCTGGAGCAAACTTGGTAGAGAATAAAGTTA
TTGCATTTAATAGTGGTGATGCTGAAAATGGACTTGGAACAATATCAGAAATTTGTTTT
CAACCGCTTTTAACTTATAAATTTGGAGATTATTTTCAGGACCCTGCACTAGATTTATT
AAAGTGTTTAATAAAATCCTTATATTATTTGTATGGAATTAAAGTTCCAGAAGATTTTAC
TTTACCGTATAGGTTGACGAATAATCCAGATAAGACAGAATATTCTCAGGTCAATATG
GAAGATTTATTAATATCAGGTGGTGATGATCTTAATGCTGCAGGGCAGAGACCATATT
GGCTATGGAATAATTATTTTATAGACGCAAAGGATAAATTTGATAAATATAAAGAAATT
TACGAAAACCAAATGAAACTGGATCCTAATCTAGAAATTAATCTTTCAAATCATTTAGA
GCAAAAATTTAATATAAACATATCTGAATTATGGAGCTTAAACATATCTAATTTTGCAA
GAACATTTAATTTAAAATCACCTAGAAGTTTTTATAAAGCACTTAAATATTATTATAGAA
AAAAATATTATAAGATACATTATAATGAAATATTTGGAACAAATTATAATATATATGGAT
TTATAGATGGACAAGTTAATGCATCACTAAAAGAAACTGATTTAAATATTATAAATAAA
CCACAGCAGATTATTAACCTTATTGATAATAACAATATATTATTAATAAAGTCCTATATA
TATGACGATGAATTAAATAAAATAGATTATAATTTTTATAATAATTATGAAATCCCTTAT
AACTATGGAAATTCTTTTAAAATACCTAATATAACGGGAATACTTTTACCTAGCGTAAA
TTATGAATTAATTGATAAAATACCAAAAATTGCTGAAATTAAACCTTATATTAAAGACTC
AACACCATTACCAGATTCTGAAAAAACGCCTATTCCTAAAGAGTTAAATGTAGGAATT
CCATTACCTATTCATTATTTGGATTCACAAATTTATAAAGGAGATGAAGATAAAGATTT
TATATTATCTCCTGACTTTCTAAAGGTTGTGTCCACCAAAGATAAATCTCTAGTATATA
GCTTTTTACCCAATATTGTTTCATATTTTGATGGATATGATAAAACAAAAATTTCTACTG
ACAAAAAATATTATTTATGGATAAGGGAAGTTTTAAATAATTATTCAATAGATATAACTA
GAACTGAAAATATAATTGGTATTTTTGGAGTAGATGAGATAGTTCCTTGGATGGGAAG
GGCCTTGAATATCTTAAATACAGAAAATACTTTTGAAACTGAACTTAGAAAAAATGGCT
TAAAAGCTTTGCTTTCTAAAGATTTAAACGTTATTTTCCCAAAAACAAAAGTGGATCCA
ATACCTACAGATAATCCTCCCCTTACAATAGAAAAAATAGATGAAAAACTTTCAGATAT
TTATATTAAAAATAAATTCTTTTTAATAAAAAATTACTACATAACTATACAGCAATGGTG
GATATGTTGCTATAGTCAATTTTTAAATCTTAGTTATATGTGTCGTGAAGCAATAATAA
ATCAACAAAATTTAATTGAAAAAATTATTTTAAATCAACTCAGCTATTTAGCTCGTGAG
ACAAGCATTAACATAGAAACGTTGTATATATTAAGTGTAACAACTGAAAAGACAATAGA
AGATTTAAGAGAAATATCACAAAAGTCAATGAATAATATATGCAATTTTTTTGAACGAG
CTAGTGTTTCAATATTCCATACTGATATTTACAATAAGTTTATTGATCATATGAAATATA
TAGTTGATGATGCAAATACTAAGATTATAAATTATATAAATTCTAATTCTAATATTACAC
AAGAAGAAAAAAATTACTTAATTAATAAATATATGCTAACAGAAGAAGATTTTAATTTTT
TCAATTTTGATAAATTAATAAATTTATTTAATTCTAAAATTCAACTCACAATTAAAAATGA
AAAGCCGGAATATAATTTATTACTATCTATAAATCAAAATGAGAGTAATGAGAATATTA
CCGATATATCAGGAAATAATGTAAAAATTAGTTATTCAAATAATATTAACATATTAGATG
GCAGAAATGAACAGGCAATATATTTAGATAATGATAGTCAATATGTTGACTTCAAATCT
AAAAATTTTGAAAATGGAGTAACTAATAATTTTACAATTAGTTTTTGGATGAGAACTTTA
GAGAAAGTAGACACAAATTCTACATTGTTAACATCTAAACTTAATGAGAATTCTGCAG
GATGGCAACTGGATTTAAGAAGAAATGGATTAGTTTGGAGTATGAAAGATCACAACAA
AAATGAAATAAATATTTATTTAAATGATTTTTTAGATATAAGTTGGCACTATATCGTTGT
TTCAGTTAATCGTTTAACAAATATATTAACTGTATATATAGATGGTGAGCTTAGTGTTA
ACAGAAATATTGAGGAAATATATAATCTATATTCAGATGTGGGGACAATTAAACTGCA
AGCAAGTGGATCTAAAGTTCGCATTGAATCTTTTTCGATTTTAAACAGAGACATTCAAA
GAGATGAGGTATCTAATAGATACATTAATTATATTGATAATGTAAATTTAAGGAATATA
TATGGGGAGAGATTAGAATACAACAAGGAATATGAAGTATCTAATTATGTTTATCCTA
GAAACTTACTATACAAGGTCAATGATATATATTTAGCTATTGAGAGAGGAAGCAACAG
TTCTAACAGGTTTAAATTAATATTAATAAATATAAATGAAGATAAAAAATTTGTACAGCA
AAAAGACATAGTTATTATTAAAGATGTCACTCAAAATAAATATTTAGGTATTTCAGAAG
ATAGTAATAAGATTAAGCTAGTAGATAGAAATAATGCTTTAGAGTTGATTCTAGATAAT
CATCTTCTTAATCCTAATTATACGACATTTTCTACTAAACAAGAAGAATATTTAAGACTA
TCTAATATAGATGGAATATATAACTGGGTGATAAAGGATGTATCGAGATTAAATGATAT
ATATTCTTGGACTTTAATATAAACTATTAAAAATTTTAAAATAAGGAGGTTGTATCAACT
TCAAATGCATGCTAATCAATGTTTAATACATTAGAAATTAGAAGGGGGGGGTAAGATG
AATAGGGAGTTTCCATTCCATTTTAATGATGGGAATGTTTCGATGAATGGATTATTTTG
TTTAAAGAAAATAAAAACGCAATATCATCCAAATTATGATTATTTCAAAATTAAATTCTG
TGAAGGGTTTTTATCTATAAAGAATAAGGTTAAAGATGATTTGTGTGAATATGATTTGA
AAAACATTGAATCCGTAATTGCATTAAAAAGAGAATATTCAAAAGAAAATAATTTAAAA
AATAAAGAATCAGCAATTTTTATGAATATTGGGAATAAAGGGATTCATAATAAATATGA
TTTATATGTTGTAAATGTAGATATTAACAATATTTTAGATGAAAATTATATGTTAAAAGG
AATATTAAATGATAAGCTAAAGATTCTTTTTTTAGGTAATGAAAGGAAGTTATTAAGAA
TAAAAAATTAGGGGGAGGAATTATGAGTAAAAAACCATTAGATTTTCTAAGAATTTATG
ATTGGCATAAAACTGAAGCAATGAACAAAATTAGTAAACTAGATTTTGAAAGGATAATT
CCTAAACATTTTTCAAAAGAAATTAAAAATAAACACTTAAGTGTTAAAATTACTGGTAA
CTGGAAAATTTGGAAGTTAACAGATGAAGGAGAAGGGCAATATCCTATTTTTAAATGC
ATAGTTGAAGATGGATTCTTAAAAATAAAAAATGAATGTGGAAATAAAAAATATTCACT
AGATAATGCTTGGATAAAAATTTGTACAAAAATTAAATATGATAATGAAAATGGAAAAG
ATATCTATTCAATAGATGAAAAAAACTTAACATTGTACAGTGTTAATAATTCATTTAACT
CAAAATATAAAAATAATATTGTAGATGCTTTTTTAGATAATTTATTAATAGCGTGTATTG
AGGACAATATAAAAGATTTAAATAAGTTTTTTAAGCTATATAAAGTTAAAACAGCAATA
AAAGAAGATTTAAGTCTCTTAGGATGGGATACAGGATACTCAACATCATTTACTCATG
TAAATAAAACTATTGAAAATCAACAGAATTATCCGAAGCAGTTTAAATATGAGTCTGAG
GGTCCTTATAACATTGATATATCTGGAGAATTTGATTCATGGAGATTAACTACTGGATC
AGATGGTCAAAATGTTAATTTTATTTGTCCAATTAAAAATGGTGAATTTAACTTTTTGG
GAACCGAGTATAAATTTTCACAAGGTGAACAAGTTAATATACAACTTAAGTTAAAATAT
TTAAATATTGAAGAGCCAACCTTTGAAGATTCAACTTCCTTAAATGATGGAAATCAGGT
TGATTTAATTGTTAAAACAGATGAAGACGAGAATGAAAATCCTCCGGTTACAATTATAA
AAGTAGTTTTACTAGGTGAAATTGACGCTATTGGTAAGATGCTTTTAGAGGGTACGTT
TAGAGAGTGGTTTAATGAAAATATTGATGCATTTAAACAAATATTTTCTTCTTTCCTTTT
AGAGGATACATCTAAAAATCCAGATTTTCAGTGGTTAAAACCTACAAAGGCTTATTAT
GGAGTTGCAAGTGCTGAACCAATAGACGGAAAGCCTGACTTAGATAGTAGTGTATTT
TCTGTCATGTCTATGGTAGAAGATAATAAAAATGATAAACCAAGTCATACAGTAGATG
GTAGAATACTTGATGCTGTTAATAATGAATCTGCATTTGGAATTAGAACCCCATTATTT
GTTAAAAAATGGCTTATTGCCGGACTAGAAATGATGCAAATTGGAAAATTAGAAGATT
TTGATTTAATAAATAACGGAATGGGATTTATTAATAACAAGAAACTTTTGTTTGGTACT
TTTGAAAATGCTGATGGTGAAGATGTACCTGCTTATGTAGAAAAAGATAATTTTAGATT
AGAAATAACGAATAATCAACTAAAAATAGAAATAACAGATATATATTGGCAGCAATCAA
GAAGATTAACAGGGCATGTAATGTATAGCCAATATTTTGATTTAGAATTAAGAAGCGG
AACTGATATCACTGGAGCAGAATATAAAAATATTTTAATTCCAGTAGAAAATTCAGAGC
CAACATTGGTAGTAAACATTTCACAAGATGAATTTGATATTTGGGGAGATATTGTCGG
TGAAATAGTTGGAGGTATAGTTGTGGGAATAGTCACAGGTTACTTAGGTAGCATTTTA
GGCAAAGGAGTAGGAAAATATTTAGAAAAATTCCTTACAAAAACATCTGGTGGAAGAT
GGGTATTAAAAATGAATAAAGAGATGTATGATTATTTAAATAATTTATTTAAAGGAGAT
AGAAGAGTTTTCAATGAAGTTGCCATAGATGAAATAGAACTGATTTCAACATTAGGAA
CATCTCAAGCTATATCAACAATTGCAAATACACCTACTAATTTTGCATCTAAAATATGG
GTAAATAAATCAAAATTTATAGGTGGTTTAATTGGGGGGTCAGTAGGCTCAGTAATAC
CTAGCGTTATTATAAAATCAATAGACGCTTGGGATAAACAAAATTATTCTGTTCTTCCA
AGTATAAATGCATTTGTAGCTTCAAGTGTAGGTTCTGTAAAATGGCCGGATACCAGTG
AATTCAAGATTGAATCAGCTGAGCTTAACGGAATTTTTTTGTTAGGTGGAAAGCTAGA
AAGATATGAAAAATAATAGAATAAAAGGATAATAATAAAAAGATAAGATAGAAAAATTT
GTCTTATCTTTTTATAAATATAGTTTGAAAGGGGAATTTAAACTATGATAGGAAAACGT
CAAACAAGTACACTGAATTGGGATACAGTATTTGCTGTTCCTATTAGTGTAGTAAATAA
AGCGATAAAAGATAAAAAAAGTAGCCCTGAGAATTTTGAATTTGAAGATTCATCTGGT
AGTAAATGTAAAGGGGATTTTGGAGATTGGCAAATAATTACTGGTGGTGATGGAAGTA
ATATACGAATGAAAATTCCTATTTACAATTTTAAAGCTGAACTGGTCGATGATAAATAT
GGAATTTTTAATGGAAACGGTGGATTTGAATCTGGAGAAATGAATATTCAAGTTAAGC
TTAAGTATTTTCCACATGATAAAATATCAAAATATAAAGATGTTGAATTAGTTGATTTAA
AAGTAAGATCAGAAAGTGCTGATCCAATTGATCCAGTAGTAGTTATGCTCTCATTGAA
GAATTTAAATGGGTTTTATTTTAATTTTTTAAATGAATTTGGTGAAGATTTACAAGATAT
TATAGAGATGTTTTTTATAGAGCTCGTTAAACAATGGCTGACAGAAAATATTAGTTTAT
TTAACCATATTTTTAGTGTAGTAAACTTAAATTTATATATTGATCAATATTCTCAATGGT
CATGGAGTAGGCCTTCATATGTTAGCTATGCTTATACAGATATAGAAGGTGATTTAGA
TAAAAGTCTATTAGGGGTTTTGTGTATGACAGGAGGAAGAAATCCTGATCTTAGACAA
CAGAAGGTAGATCCTCATGCAGTACCAGAAAGTTCTCAATGTGGATTTTTAATTTATG
AAGAGAGGGTATTAAGAGATTTACTTTTACCAACTTTACCAATGAAATTTAAAAATTCA
ACAGTAGAAGATTATGAGGTAATTAATGCAAGCGGAGAAAGTGGTCAGTATCAGTATA
TATTAAGATTAAAAAAAGGTAGGAGTGTTAGTTTAGACCGCGTTGAGGCTAATGGTTC
TAAATATGATCCATATATGACTGAAATGAGTATTAGTTTATCAAATGATGTATTAAAACT
AGAAGCAACCACAGAAACTTCGGTAGGAATGGGAGGAAAAGTTGGATGTGATACTAT
AAATTGGTATAAGTTAGTACTTGCAAAAAATGGAAATGGAGAACAAACTATATCATATG
AAGAAGTTGGAGAACCTACAGTAATAAATTATGTAATAAAAGAAGGCGAAAATTGGGT
ATGGGATGTAATCGCTGCAATCATAGCTATTCTAGCAACAGCAGTATTGGCAATATTT
ACTGGAGGAGCAGCTTTTTTTATAGGTGGTATTGTTATAGCTATAATAACAGGATTTAT
AGCTAAAACTCCAGATATAATTTTAAATTGGAACCTTGAAACTTCTCCAAGTATAGATA
TGATGTTAGAAAATTCTACTTCACAAATTATTTGGAATGCTAGAGACATATTTGAACTA
GATTATGTTGCTTTAAATGGACCACTGCAACTAGGTGGAGAATTAACTGTTTAAAATTA
AAAATTTTAATAAGAATAATTTTTATATATTTATTATAGATACCTTAAAGGAGTAGGGAA
ATGTATGCTACAAATAAGAGTTTTTAATTATAATGATCCAATTGATGGAGAAAATATCG
TGGAGTTAAGATACCATAACAGGAGCCCTGTAAAAGCATTTCAAATAGTAGATGGTAT
ATGGATAATTCCAGAAAGATATAACTTTACAAACGATACAAAAAAAGTTCCAGACGAT
CGAGCTCTTACTATTCTGGAAGATGAAGTTTTTGCTGTTCGCGAAAATGACTATTTAA
CAACAGATGTTAATGAAAAAAATTCCTTTTTAAATAATATTACTAAGCTTTTTAAGCGTA
TTAATTCAAGTAACATTGGTAATCAGTTACTTAATTATATTTCAACAAGCGTCCCATAT
CCAGTTGTGAGTACAAATTCAATAAAGGCTAGAGACTATAATACAATTAAATTTGATTC
AATTGATGGGCGAAGAATTACAAAATCTGCAAATGTACTTATCTACGGACCAAGTATG
AAAAATTTACTAGATAAACAAACAAGGGCTATCAATGGGGAAGAAGCAAAAAATGGTA
TAGGATGTTTAAGTGATATTATTTTTTCTCCAAATTACTTATCTGTCCAAACTGTTTCTT
CAAGTAGGTTTGTTGAAGATCCTGCATCATCACTTACACATGAACTTATCCATGCCTT
ACATAATTTATATGGAATACAATATCCTGGAGAAGAAAAATTTAAATTTGGAGGATTTA
TTGATAAACTATTAGGAACTAGAGAATGCATAGATTATGAGGAAGTCTTAACATATGG
AGGAAAAGATTCCGAAATTATAAGAAAGAAAATTGATAAGTCCTTATATCCTGATGATT
TTGTAAATAAGTATGGTGAAATGTATAAGCGTATAAAAGGATCTAATCCTTATTATCCC
GACGAAAAAAAATTAAAACAAAGTTTTTTAAACAGAATGAATCCATTTGATCAAAATGG
AACTTTTGATACTAAAGAATTTAAAAATCATCTTATGGATTTATGGTTTGGGTTAAATG
AGAGTGAATTTGCTAAAGAAAAGAAGATTTTAGTCAGAAAGCACTATATAACAAAGCA
AATTAATCCTAAATACACAGAACTTACTAATGATGTATATACTGAAGATAAAGGCTTTG
TAAATGGTCAATCTATAGACAATCAAAATTTTAAAATAATTGATGATTTAATATCAAAAA
AAGTAAAACTATGTTCTATAACATCTAAAAATCGAGTAAATATTTGTATAGACGTTAAT
AAAGAAGATTTATATTTCATAAGTGATAAAGAAGGTTTTGAAAATATAGATTTTTCCGA
GCCGGAAATTAGATATGATAGTAATGTAACTACAGCAACTACCTCTTCTTTTACAGAC
CATTTTTTAGTAAATAGAACTTTTAACGATAGTGATAGATTTCCACCTGTAGAATTAGA
ATATGCTATCGAACCAGCTGAAATAGTTGATAACACTATAATGCCAGATATTGATCAA
AAAAGCGAAATATCTCTCGATAACTTAACGACCTTTCACTATTTAAATGCTCAAAAAAT
GGATTTGGGATTTGATTCATCAAAAGAACAGTTAAAGATGGTTACATCAATAGAGGAA
TCATTATTAGATTCAAAAAAGGTATACACACCATTTACGAGAACTGCACATAGTGTAAA
TGAACGTATATCTGGAATAGCGGAAAGTTACTTATTTTATCAATGGTTAAAAACTGTTA
TAAATGATTTTACAGATGAATTAAACCAAAAGAGTAATACTGACAAAGTTGCTGATATT
TCTTGGATTATACCCTATGTTGGACCTGCTTTAAATATTGGCCTTGATTTATCTCATGG
AGATTTTACTAAAGCTTTTGAAGATTTAGGGGTTTCTATTTTATTTGCTATTGCTCCAG
AATTTGCAACTATAAGTCTTGTAGCTCTTTCAATATATGAAAATATAGAAGAGGATTCA
CAAAAAGAAAAAGTAATTAATAAAGTAGAAAATACATTAGCAAGGAGAATAGAAAAAT
GGCACCAAGTTTATGCTTTCATGGTGGCTCAGTGGTGGGGTATGGTTCATACTCAGA
TAGACACTAGAATTCATCAAATGTATGAATCACTTTCTCATCAAATTATAGCAATTAAA
GCTAATATGGAGTATCAGTTATCTCATTATAAAGGCCCTGATAATGATAAACTTCTATT
AAAGGATTATATATATGAGGCTGAAATAGCTCTTAACACTTCAGCAAATCGAGCAATG
AAAAATATTGAAAGATTTATGATTGAAAGCTCTATTTCATACTTAAAAAATAATCTAATT
CCCAGTGTAGTAGAAAATTTAAAAAAATTTGATGCTGATACAAAAAAGAATTTAGATCA
ATTTATTGATAAAAATTCCTCAGTATTAGGATCTGATTTACATATATTAAAGTCTCAAGT
AGATTTAGAACTTAATCCAACTACTAAGGTAGCCTTTAATATTCAAAGTATTCCAGATT
TTGATATAAATGCATTGATAGACAGATTAGGTATTCAATTAAAAGATAACTTAGTATTT
AGTTTAGGAGTGGAATCTGATAAAATAAAAGATCTATCTGGGAATAATACAAACCTAG
AAGTTAAAACAGGTGTCCAAATAGTAGATGGACGAGATAGTAAGACTATACGTTTAAA
TTCAAATGAAAATTCAAGTATTATAGTTCAGAAAAATGAAAGTATAAACTTCTCATATTT
TAGTGACTTTACCATAAGTTTTTGGATAAGAGTTCCAAGACTTAATAAAAATGATTTTA
TAGACTTAGGAATTGAATATGACTTAGTAAATAATATGGATAATCAAGGATGGAAAATT
TCGCTTAAGGATGGGAATTTAGTATGGAGAATGAAAGATAGATTTGGAAAAATAATAG
ATATTATTACGTCTTTAACCTTTAGTAATAGCTTTATAGATAAATATATATCCAGTAATA
TATGGAGACATATAACTATTACAGTTAACCAATTAAAAGATTGTACTTTATATATAAATG
GAGATAAAATAGATAGTAAATCAATTAACGAATTAAGAGGTATCGATAATAATTCTCCA
ATAATATTCAAGTTAGAAGGGAATAGAAATAAAAATCAATTTATACGCTTAGATCAGTT
TAATATTTATCAAAGGGCTTTAAATGAAAGTGAAGTTGAAATGTTATTTAATAGTTATTT
TAATTCAAATATATTAAGAGATTTTTGGGGAGAACCTTTAGAGTATAATAAGAGTTACT
ATATGATAAATCAAGCAATATTAGGTGGACCCCTTAGAAGCACATATAAGTCATGGTA
TGGAGAGTATTACCCTTATATATCTAGAATGAGGACGTTTAATGTTTCATCATTTATTT
TAATTCCTTACCTATATCATAAAGGATCAGATGTAGAAAAGGTAAAAATAATAAATAAA
AACAACGTGGATAAATATGTAAGAAAAAATGATGTAGCAGATGTTAAATTTGAAAATTA
TGGTAATTTAATACTTACGTTACCTATGTACAGTAAAATCAAAGAGAGATATATGGTAT
TAAACGAGGGTAGAAACGGCGATTTAAAGTTAATTCAATTACAAAGTAACGATAAATA
CTATTGTCAAATACGAATATTTGAAATGTACAGAAATGGGTTGCTGTCAATTGCAGAC
GATGAAAACTGGTTATACTCTAGTGGCTGGTATTTATACTCTAGTGGCTGGTATTTAG
ATAATTATAAAACTTTGGATTTAAAAAAACATACAAAAACTAATTGGTATTTTGTTAGTG
AAGATGAAGGATGGAAGGAATAG

While the present disclosure has been illustrated and described with reference to certain exemplary embodiments, those of ordinary skill in the art will understand that various modifications and changes may be made to the described embodiments without departing from the spirit and scope of the present disclosure, as defined in the following claims.

What is claimed is:

1. A composition comprising a microbe genetically modified to express a heterologous clostridial mosquitocidal protein 1 (CMP1) protein having an amino acid sequence of SEQ ID NO: 1 or a variant thereof and a heterologous non-toxic non-hemagglutinin (NTNH) protein having an amino acid sequence of SEQ ID NO: 3.

2. The composition of claim 1, wherein the microbe is not Clostridium bifermentans malaysia or Clostridium bifermentans paraiba.

3. The composition of claim 1, wherein the microbe is a bacterium, virus, yeast, or fungi.

4. The composition of claim 3, wherein the bacterium is selected from Lysinibacillus or Bacillus.

5. The composition of claim 4, wherein the Lysinibacillus bacterium is Lysinibacillus sphaericus and the Bacillus bacterium is Bacillus thuringiensis.

6. The composition of claim 1, wherein the microbe also expresses a heterologous OrfX1 protein having an amino acid sequence of SEQ ID NO: 5, a heterologous OrfX2 protein having an amino acid sequence of SEQ ID NO: 7, and/or a heterologous OrfX3 protein having an amino acid sequence of SEQ ID NO: 9.

7. The composition of claim 6, wherein the microbe is genetically modified with a nucleic acid vector comprising an operon encoding ntnh-orfX1-orfX2-orfX3-cmp1.

8. The composition of claim 7, wherein the operon has a nucleic acid sequence of SEQ ID NO: 11.

9. The composition of claim 1, wherein the variant thereof is a homolog of the CMP1 protein having at least 85% identity with SEQ ID NO: 1 and capable of aligning with amino acid residues S1095, W1096, Y1097, and G1098 of SEQ ID NO: 1.

10. The composition of claim 1, wherein the variant thereof is a homolog of the CMP1 protein having at least 95% identity with SEQ ID NO: 1 and capable of aligning with amino acid residues S1095, W1096, Y1097, and G1098 of SEQ ID NO: 1.

11. A nucleic acid expression vector comprising a nucleic acid sequence encoding for a clostridial mosquitocidal protein 1 (CMP1) protein having an amino acid sequence of SEQ ID NO: 1 and a nucleic acid sequence encoding for a non-toxic non-hemagglutinin (NTNH) protein having an amino acid sequence of SEQ ID NO: 3.

12. The nucleic acid expression vector of claim 11, capable of being transformed into a bacterium, virus, yeast, or fungus.

13. The nucleic acid expression vector of claim 11, further comprising a nucleic acid sequence encoding for an OrfX1 protein having an amino acid sequence of SEQ ID NO: 5, an OrfX2 protein having an amino acid sequence of SEQ ID NO: 7, and/or an OrfX3 protein having an amino acid sequence of SEQ ID NO: 9.

14. The nucleic acid expression vector of claim 11, wherein the nucleic acid sequence is an operon encoding for NTNH having an amino acid sequence of SEQ ID NO: 3, ORFX1 having an amino acid sequence of SEQ ID NO: 5, ORFX2 having an amino acid sequence of SEQ ID NO: 7, ORFX3 having an amino acid sequence of SEQ ID NO: 9, and CMP1 having an amino acid sequence of SEQ ID NO: 1.

15. A method of decreasing a population of an Anopheles mosquito species, comprising administering or exposing the composition of claim 1 to the Anopheles mosquito species.

16. The method of claim 15, wherein the Anopheles species is selected from Anopheles gambiae, Anopheles coluzzi, Anopheles funestus, Anopheles darlingi, or Anopheles stephensi.

17. The method of claim 15, wherein the microbe is a bacterium is selected from Lysinibacillus or Bacillus.

18. A method of decreasing a population of an Anopheles mosquito species, comprising administering or exposing the composition of claim 6 to the Anopheles mosquito species.

19. The method of claim 18, wherein the microbe is a bacterium is selected from Lysinibacillus or Bacillus.

20. A method of killing an Anopheles mosquito species comprising injecting a composition comprising a CMP1 protein having an amino acid sequence of SEQ ID NO: 1 or a variant thereof to the Anopheles mosquito species.
